(19) United States
(12) Patent Application Publication
(10) Pub. No.: US 2020/0043582 A1
(43) Pub. Date: Feb. 6, 2020

(54) IMMUNIZATION TRACKING AND NOTIFICATION METHODS AND SYSTEMS

(71) Applicant: IMMUTRACK LLC, Metairie, LA (US)

(72) Inventor: Sophia L. Riviere, Metairie, LA (US)

(73) Assignee: IMMUTRACK LLC, Metairie, LA (US)

(21) Appl. No.: 16/054,873

(22) Filed: Aug. 3, 2018

(51) Int. Cl. G16H 10/65; G06Q 10/06; G16H 20/10; G06Q 10/10; G16H 80/00

(52) U.S. Cl. CPC G16H 10/65; G06Q 10/06; G16H 80/00; G06Q 10/109; G16H 20/10

(57) ABSTRACT

A computer implemented method and system for tracking immunization is provided. The computer implemented method includes steps of receiving one or more records corresponding to one or more respective family members, from a client device, storing the one or more records in a data repository, analyzing the one or more records for generating one or more recommendations and transmitting the one or more generated recommendations to the client device.

### BACKGROUND OF THE INVENTION

### Field of the Invention

The present invention generally relates to medical health information management and more particularly to a computer implemented method and a computer system for tracking immunization.

### Description of the Related Art

With the increasing demand for people's health, requirements for immunization are also increasing. Immunization demands proper and strict schedule arrangements for better treatment outcomes. In order to ensure suitable and timely immunization, it is required to stay updated about the vaccination regimes associated with an individual. The process of inquiring about vaccine status or obtaining hard-copies of vaccine records via a traditional route requires direct interaction with the medical office, either face-to-face or by telecommunication, to retrieve the documentation. This interaction with healthcare provider can be cumbersome and inconvenient for an individual, often requiring an individual to miss days of work and endure travel expenses and possibly day care expenses, as well as placing additional workloads on busy clinic staff. Furthermore, when clinic staff shift their focus towards these tasks, overall access to healthcare for clinic patients is delayed due to multiple interruptions in a normal clinic flow.

In today's busy life-style, it is difficult to keep track of immunization or vaccination schedules. Lack of access to comprehensive, evidence-based information about vaccinations and lack of knowledge about vaccination schedules and status are barriers to immunization uptake. Furthermore, there are many impediments against immunization, including misinformation about vaccines, adverse effects of vaccines, vaccine-preventable diseases and disease development after the administration of vaccines. Hence, inadequate knowledge about the adverse effects and contraindications of vaccines often lead to many immunization errors.

All the above mentioned reasons and circumstances can amplify the problems associated with avoiding immunization or vaccination, and makes the task of maintaining one's own health care and health care information burdensome and inefficient. Through conventional systems and methods, it is difficult to determine a comprehensive immunization status for a particular individual and that of their family members, and also, patients have difficulty in understanding their routine and preventive healthcare needs. Moreover, patient access to their own immunization records can be complex, time-consuming, and expensive. Yet further, a parent may be unable to view immunization information for his or her child in a common system.

In light of the discussion above, there is need for a computer implemented method and a computer system for tracking immunization, which alleviates one or more of the above mentioned deficiencies.

Any discussion of the background art throughout the specification should in no way be considered as an admission that such background art is prior art nor that such background art is widely known or forms part of the common general knowledge.

As disclosed in this application, the inventor has discovered novel and unique systems and methods for efficient and comprehensive tracking, cross-referencing, and notification of immunization records, requirements, health conditions, and immunization contraindications.

Embodiments of the present invention provide for devices and methods as disclosed herein and as defined in the annexed claims which provide for improved immunization tracking, cross-referencing, and notification features in order to efficiently track and inform people of immunization shots and vaccination requirements, for people's health and well-being.

### SUMMARY OF THE INVENTION

It is one prospect of the present invention to provide one or more novel systems of simple but effective implementation which can be applied in many environments to efficiently and effectively provide for tracking immunizations.

The following presents a simplified summary of the present disclosure in a simplified form as a prelude to the more detailed description that is presented herein.

Therefore, in accordance with embodiments of the invention, there is provided a computer implemented method for tracking immunization. The computer implemented method comprises steps of receiving one or more immunization records from a state immunization registry, each immunization record corresponding to one or more respective persons; receiving one or more schedules of vaccination requirements; storing the one or more immunization records and schedules of vaccination requirements in a data repository; receiving a request requesting notification of vaccination recommendations, from a client device; analyzing, using a computer processor, the one or more immunization records by cross-referencing the one or more immunization records with the one or more schedules of vaccination requirements; generating one or more notifications of vaccination recommendations in response to receiving the request; and transmitting the one or more notifications of vaccination recommendations, to the client device.

In another embodiment, the computer implemented method further comprises the step of tracking a survival or disease status of a user taking action toward achieving goals associated with improving health based upon the at least one notification of vaccination recommendations.

In yet another embodiment, the computer implemented method for tracking immunization further comprises receiving one or more user vaccination records from the client device, and the step of analyzing the one or more immunization records includes cross-referencing the one or more user vaccination records with the one or more schedules of vaccination requirements, for generating the one or more notifications of vaccination recommendations in response to receiving the request.

In one embodiment, the one or more schedules of vaccination requirements in the computer implemented method for tracking immunization includes seasonal vaccination requirements, such that the request for notification of vaccination recommendations is automatically triggered seasonally based upon the seasonal vaccination requirements. For example, the request for notification of vaccination recommendations is automatically triggered every flu season, such that the computer notifies a user of the recommendation to get the flu vaccine.

In yet another embodiment, the computer implemented method includes receiving one or more user date of birth records from the client device, and the step of analyzing the one or more immunization records includes cross-referencing the one or more user date of birth records with the one or more schedules of vaccination requirements, for generating the one or more notifications of vaccination recommendations in response to receiving the request. In another embodiment, the request for notification of vaccination recommendations is automatically triggered annually, based upon the date of birth.

In another embodiment, the computer implemented method includes receiving one or more user life event records from the client device, and the step of analyzing the one or more immunization records includes cross-referencing the user life event records with the one or more schedules of vaccination requirements, for generating the one or more notifications of vaccination recommendations in response to receiving the request. In yet another embodiment, the request requesting notification of vaccination recommendations is automatically triggered based upon the life event.

In one embodiment of the computer implemented method of immunization tracking, the user life event records may include, for example, childbirth. In such exemplary embodiment, the one or more schedules of vaccination requirements includes a Tdap vaccine requirement to protect newborn children from whooping cough, and the request for notification of vaccination recommendations is automatically triggered when the user life event records includes childbirth.

In a preferred embodiment, the computer implemented method of immunization tracking includes receiving one or more geographical area outbreak records (i.e., outbreak information) associated with vaccine preventable diseases, from a third party device, and the step of analyzing the one or more immunization records includes cross-referencing the one or more geographical area outbreak records with the one or more schedules of vaccination requirements, for generating the one or more notifications of vaccination recommendations in response to receiving the request.

In one such embodiment, the computer implemented method further comprises receiving at least one user geographic location record of information as provided by a client device having an on-board GPS, and the request for notification of vaccination recommendations is automatically triggered when the one or more geographical area outbreak records includes the user geographic location record. For example, the one or more geographical area outbreak records may include, for example, an outbreak of measles in the city of New Orleans, so when the computer receives at least one user geographic location record from the client device indicating that the geographic location of the client device is located within the city of New Orleans, then the request for notification of vaccination recommendations is automatically triggered. In such example, the request for notification of vaccination recommendations is automatically triggered because the location of the client device is located within the geographical area of the outbreak, which is provided to the computer as part of the geographical area outbreak records. Embodiments includes cross-referencing the geographical location of the user's device and the current geographical area of the outbreak of a vaccine preventable disease.

In yet another embodiment, the computer implemented method further includes receiving one or more user allergy records from the client device, and the step of analyzing the one or more immunization records includes cross-referencing the one or more user allergy records with the one or more schedules of vaccination requirements, for generating the one or more notifications of vaccination recommendations in response to receiving the request.

In one embodiment, the computer implemented method of immunization tracking further includes receiving one or more user health condition records from the client device, and the step of analyzing the one or more immunization records includes cross-referencing the one or more user health condition records with the one or more schedules of vaccination requirements, for generating the one or more notifications of vaccination recommendations in response to receiving the request.

In one preferred embodiment, the schedules of vaccination requirements include vaccination requirements for a specific country of destination of a user. In another embodiment, the schedules of vaccination requirements include vaccination requirements for a school, place of employment, State, or territory of a user.

Preferably, the computer implemented method of immunization tracking includes a step of transmitting information related to the one or more recommendations, from a third party device. In one embodiment, the information includes risks and benefits related to the one or more recommendations.

In one embodiment, the computer implemented method further comprises a step of providing a schedule for vaccinations on the client device, and the schedule for vaccinations are received from a third party device. In one such embodiment, the schedule for vaccinations is designed to include schedules for one or more age groups. Preferably, the one or more age groups are selected from a group consisting of 0-6 years, 7-18 years, adults by age and adults by health condition, and the one or more recommendations includes contraindications.

In a preferred embodiment, the persons are one or more respective family members, and the computer implemented method includes a step of receiving one or more immunization records corresponding to one or more respective family members, from a client device, storing the one or more records in a data repository, analyzing the one or more records for generating one or more recommendations and transmitting the one or more generated recommendations, to the client device.

In yet another preferred embodiment, the one or more respective persons are family members, and the method includes a step of receiving details of vaccinations already received by the one or more respective family members and adding the details to the one or more immunization records corresponding to the one or more respective family members. In one embodiment, the method includes a step of updating the one or more immunization records of the one or more vaccines based on the one or more recommendations.

Preferably, the method further includes a step of providing information related to one or more vaccines, from a third party device. In another embodiment, the method includes a step of providing information related to one or more diseases prevented by respective one or more vaccines, from a third party device. Preferably, the information includes a disease name, a vaccine name, disease symptoms, disease causes and disease complications.

In another embodiment, the method further includes a step of providing information related to one or more vaccine exemptions, from a third party device.

In accordance with an embodiment of the present invention, each record of the one or more records, preferably comprises a name, a date of birth (DOB), a gender, a birth state, medical history and allergies.

In accordance with an embodiment of the present invention, the computer implemented method further comprises a step of providing requirement for vaccination on the client device, the requirement for vaccination being provided based on one or more states corresponding to the one or more respective family members.

In accordance with an embodiment of the present invention, the one or more recommendations is selected from a group consisting of medicine, therapy, counseling and vaccination.

In accordance with an embodiment of the present invention, the computer implemented method further comprises a step of notifying the client device for vaccinations corresponding to the one or more respective family members.

In accordance with an embodiment of the present invention, the computer implemented method further comprises a step of notifying the client device for upcoming vaccinations corresponding to the one or more respective family members.

In accordance with an embodiment of the present invention, the computer implemented method further comprises a step of notifying the client device for due vaccinations corresponding to the one or more respective family members.

In accordance with an embodiment of the present invention, the computer implemented method further comprises a step of notifying the client device for vaccinations based on one or more dates of birth (DOB), corresponding to the one or more respective family members.

In accordance with an embodiment of the present invention, the computer implemented method further comprises a step of transmitting information related to the one or more recommendations, from a third party device.

In accordance with an embodiment of the invention, the information includes risks and benefits related to the one or more recommendations.

In accordance with an embodiment of the invention, the computer implemented method further comprises a step of providing a schedule for vaccinations on the client device, the schedule for vaccination being received from a third party device.

In accordance with an embodiment of the present invention, the schedule for vaccinations is designed to include schedules for one or more age groups.

In accordance with an embodiment of the present invention, the one or more age groups are selected from a group consisting of 0-6 years, 7-18 years, adults by age and adults by health condition.

In accordance with an embodiment of the present invention, the computer implemented method further comprises a step of transmitting the one or more records corresponding to the one or more respective family members, to the client device.

In accordance with an embodiment of the present invention, the computer implemented method further comprises steps of receiving details of vaccinations already received by the one or more family members and adding the details to the one or more records corresponding to the one or more respective family members.

In accordance with an embodiment of the present invention, the computer implemented method further comprises a step of updating the one or more records of the one or more vaccines based on the one or more recommendations.

According to another aspect of the invention, there is provided a computer system for tracking immunization. The computer system is in communication with a client device associated with a user, over a network. The computer system comprises a memory unit configured to store machine readable instructions, and a processor operably connected with the memory unit, the processor obtaining the machine-readable instructions from the memory unit, and being configured by the machine-readable instructions to receive one or more records corresponding to one or more respective family members, from a client device, store the one or more records in a data repository, analyze the one or more records for generating one or more recommendations and transmit the one or more generated recommendations, to the client device.

In accordance with an embodiment of the present invention, each record of the one or more records, comprises one or more of, for example, a name, a date of birth (DOB), a gender, a birth state, medical history and allergies.

In accordance with an embodiment of the present invention, the processor is further configured to provide requirement for vaccination on the client device, the requirement for vaccination being provided based on one or more states corresponding to the one or more respective family members.

In accordance with an embodiment of the present invention, the one or more recommendations is selected from a group consisting of medicine, therapy, counseling and vaccination.

In accordance with an embodiment of the present invention, the processor is further configured to notify the client device for vaccinations corresponding to the one or more respective family members.

In accordance with an embodiment of the present invention, the processor is further configured to notify the client device for upcoming vaccinations corresponding to the one or more respective family members.

In accordance with an embodiment of the present invention, the processor is further configured to notify the client device for due vaccinations corresponding to the one or more respective family members.

In accordance with an embodiment of the present invention, the processor is further configured to notify the client device for vaccinations on one or more date of birth (DOB), corresponding to the one or more respective family members.

In accordance with an embodiment of the present invention, the processor is further configured to transmit information related to the one or more recommendations, to the client device, received from a third party device.

In accordance with an embodiment of the present invention, the information preferably includes risks and benefits related to the one or more recommendations.

In accordance with an embodiment of the present invention, the processor is further configured to provide a schedule for vaccinations on the client device, the schedule for vaccinations being received from a third party device.

In accordance with an embodiment of the present invention, the schedule for vaccinations is designed to include schedules for one or more age groups.

In accordance with an embodiment of the invention, the one or more age groups are selected from a group including children aged 18 years or younger based on medical indications, adults 19-21 years, adults 22-26 years, adults 27-49 years, adults 50-64 years, adults 65+ years, and adults 18+ years by health condition.

In accordance with an embodiment of the present invention, the one or more age groups are selected from a group consisting of 0-6 years, 7-18 years, adults by age and adults by health condition.

In accordance with an embodiment of the present invention, the processor is further configured to transmit the one or more records corresponding to the one or more respective family members, on the client device.

In accordance with an embodiment of the present invention, the processor is further configured to provide information related to one or more diseases prevented by respective one or more vaccines, received from a third party device.

In accordance with an embodiment of the present invention, the information preferably includes a disease name, a vaccine name, disease symptoms, disease causes and disease complications.

In accordance with an embodiment of the present invention, the processor is further configured to provide information related to the one or more vaccines exemptions, received from a third party device.

In accordance with an embodiment of the present invention, the processor is further configured to receive details of vaccinations already received by the one or more family members and add the details to the one or more records corresponding to the one or more respective family members.

In accordance with an embodiment of the present invention, the processor is further configured to update the one or more records of the one or more vaccines based on the one or more recommendations.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which like numerals throughout the figures identify substantially similar components, in which:

FIG. 1 illustrates an exemplary environment of computing devices in which or with which various embodiments described herein may be implemented;

FIG. 2 illustrates a flow diagram illustrating a computer implemented method for tracking immunization, in accordance with embodiments of the invention;

FIG. 3A illustrates a snapshot of a recommended immunization schedule for children and adolescents aged 18 years or younger, in accordance with embodiments of the invention;

FIG. 3B illustrates a snapshot of a recommended catch-up immunization schedule for persons aged 4 months-18 years who start late or who are more than 1 month behind the recommended immunization schedule illustrated in FIG. 5A for children and adolescents aged 18 years or younger, in accordance with embodiments of the invention;

FIG. 3C illustrates an exemplary schedule of recommended vaccines that might be indicated for children and adolescents aged 18 years or younger based on medical indications, in accordance with embodiments of the invention;

FIG. 3D illustrates an exemplary schedule for vaccination for adults by age, in accordance with an embodiment of the present invention;

FIG. 3E illustrates a snapshot of a schedule for vaccination for adults by health condition, in accordance with an embodiment of the present invention;

FIG. 4 is a diagram of an embodiment an exemplary window of an immunization tracker application of the system, in accordance with embodiments of the invention;

FIG. 5 is a diagram of an embodiment of an exemplary window of an immunization tracker application of the system, in accordance with embodiments of the invention;

FIG. 6 is a diagram of an embodiment of an exemplary window of an immunization tracker application of the system, in accordance with embodiments of the invention;

FIG. 7 is a diagram of an embodiment of an exemplary window of an immunization tracker application of the system, in accordance with embodiments of the invention;

FIG. 8 is a diagram of an embodiment of an exemplary window of an immunization tracker application of the system, in accordance with embodiments of the invention;

FIG. 9 is a diagram of an embodiment of an exemplary window of an immunization tracker application of the system, in accordance with embodiments of the invention;

FIG. 10 is a diagram of an embodiment of an exemplary window of an immunization tracker application of the system, in accordance with embodiments of the invention;

FIG. 11 is a diagram of an embodiment of an exemplary window of an immunization tracker application of the system, in accordance with embodiments of the invention;

FIG. 12 is a diagram of an embodiment of an exemplary window of an immunization tracker application of the system, in accordance with embodiments of the invention;

FIG. 13 is a diagram of an embodiment of an exemplary window of an immunization tracker application of the system, in accordance with embodiments of the invention;

FIG. 14 is a diagram of an embodiment of an exemplary window of an immunization tracker application of the system, in accordance with embodiments of the invention;

FIG. 15 is a diagram of an embodiment of an exemplary window of an immunization tracker application of the system, in accordance with embodiments of the invention;

FIG. 16 is a diagram of an embodiment of an exemplary window of an immunization tracker application of the system, in accordance with embodiments of the invention;

FIG. 17 is a diagram of an embodiment of an exemplary window of an immunization tracker application of the system, in accordance with embodiments of the invention;

FIG. 18 is a diagram of an embodiment of an exemplary window of an immunization tracker application of the system, in accordance with embodiments of the invention;

FIG. 19 is a diagram of an embodiment of an exemplary window of an immunization tracker application of the system, in accordance with embodiments of the invention;

FIG. 20 is a diagram of an embodiment of an exemplary window of an immunization tracker application of the system, in accordance with embodiments of the invention;

FIG. 21 illustrates a snapshot of recommended immunizations for children from birth through 6 years old in accordance with an embodiment of the present invention;

FIG. 22 is a diagram of an embodiment of an exemplary window of an immunization tracker application of the system, in accordance with embodiments of the invention;

FIG. 23 illustrates a snapshot of recommended immunizations for children from 7 years old through 18 years old in accordance with an embodiment of the present invention;

FIG. 24 is a diagram of an embodiment of an exemplary window of an immunization tracker application of the system, in accordance with embodiments of the invention;

FIG. 25 illustrates a snapshot of recommended immunizations for adults by age in accordance with an embodiment of the present invention;

FIG. 26 is a diagram of an embodiment of an exemplary window of an immunization tracker application of the system, in accordance with embodiments of the invention;

FIG. 27 illustrates a snapshot of recommended immunizations for adults by health condition in accordance with an embodiment of the present invention;

FIG. 28 is a diagram of an embodiment of an exemplary window of an immunization tracker application of the system, in accordance with embodiments of the invention;

FIG. 29 is a diagram of an embodiment of an exemplary window of an immunization tracker application of the system, in accordance with embodiments of the invention;

FIG. 30 illustrates a snapshot of vaccine-preventable diseases and the vaccines that prevent them in accordance with an embodiment of the present invention;

FIG. 31 is a diagram of an embodiment of an exemplary window of an immunization tracker application of the system, in accordance with embodiments of the invention;

FIG. 32 illustrates a snapshot of vaccine requirements in accordance with an embodiment of the present invention;

FIG. 33 is a diagram of an embodiment of an exemplary window of an immunization tracker application of the system, in accordance with embodiments of the invention;

FIG. 34 is a diagram of an embodiment of an exemplary window of an immunization tracker linking school vaccination requirements, exemptions and web links in accordance with embodiments of the invention;

FIG. 35 is a diagram of an embodiment of an exemplary window of an immunization tracker and an illustration of school vaccination requirements, exemptions and web links in accordance with embodiments of the invention;

FIG. 36 is a diagram of an embodiment of an exemplary window of an immunization tracker linking web links for safety, concerns, and research in accordance with embodiments of the invention;

FIG. 37 is a diagram of an embodiment of an exemplary window of an immunization tracker linking web links for safety, concerns, and research in accordance with embodiments of the invention;

FIG. 38 is a diagram of an embodiment of an exemplary window of an immunization tracker application of the system, in accordance with embodiments of the invention; and

FIG. 39 is a diagram of an embodiment of an exemplary window of an immunization tracker application of the system, in accordance with embodiments of the invention.

### DETAILED DESCRIPTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, method, structure or manner. It should be understood that the devices, methods, processes, procedures, and techniques described herein are presently representative of various embodiments. Other embodiments of the disclosure will readily suggest themselves to such skilled persons having the benefit of this disclosure.

Usage of mobile phones is common in all age groups. Through embodiments disclosed herein, mobile phones can act as tracking device for maintaining health related concerns. Immunization is one of a most important features of the health system and regular checks on immunization and vaccination is prudent for one's health management. However, it is difficult to keep track immunization history and updates about the regular vaccinations of an individual and his family members. The shortage of time in today's busy life and culture has made it more difficult to have time for tracking immunization related information and knowledge.

Embodiments of the invention provide tracking of immunization of not only an individual's health but also of the health of family members. A computer implemented system of mobile phones is disclosed herein enabling people to keep a regular check on all the immunization related information. A third party device is preferably provided to work together with a server. The server receives information from the third party device which is then displayed on the client device.

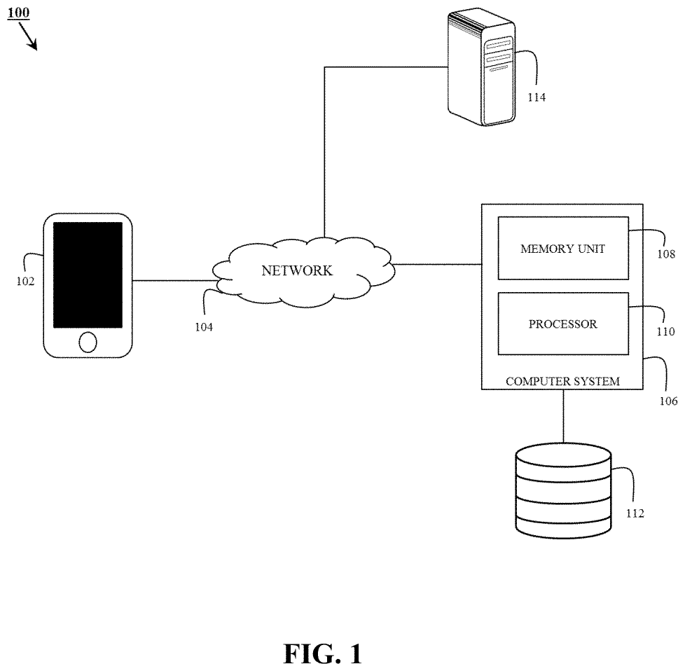

FIG. 1 illustrates an exemplary environment 100 of computing devices to which the various embodiments described herein may be implemented. As shown in FIG. 1, the environment 100 comprises a client device 102 associated with a user 103. In various embodiments, the client device 102 is preferably a mobile handheld device such as cellular phones, PDA, and tablet PC, and the like. The client device 102 is preferably wirelessly connected with a network 104. The network 104 may be implemented through one or more plurality of protocols, such as, but not limited to, Ethernet, Wi-Fi, Bluetooth, ZigBee, GSM and CDMA. Further, the network 104 may be a Local Area Network (LAN) or a Wide Area Network (WAN).

Further connected to the network 104 is a computer system 106. The computer system 106 has computing capabilities, including a memory unit 108 and a processor 110 operably connected with the memory unit 108. The memory unit 108 may be a non-volatile memory unit, such as, but not limited to, EPROM, EEPROM and Flash Memory. The processor 110 may be for example a general purpose processor, an Application Specific Integrated Circuit (ASIC) or a Field Programmable Gate Array (FPGA). The memory unit 108 is configured to store machine readable instructions. The machine readable instructions are preferably loaded into the memory unit 108 from a non-transitory computer readable medium. A data repository 112 capable of storing one or more records is connected with the computer system 106. In accordance with an embodiment, the data repository (160) processes queries in a plurality of languages including Structured Query Language (SQL), QL and Datalog. The data repository 112 is a local storage device or a networked storage device.

Also connected with the network 104 is the third party device 114. The third party device 114 transfers information to the computer system 106 which is displayed while the information is accessed on the client device 102. Further, the third party device 114 may be, for example, a State immunization registry or a regulatory body for example, Centre for disease control and prevention (CDC), Vaccine Information Statement (VIS) and Advisory Committee on Immunization Practices (ACIP) for U.S.

Various embodiments may now be understood with reference to an exemplary environment 100 shown in the Figures and described herein.

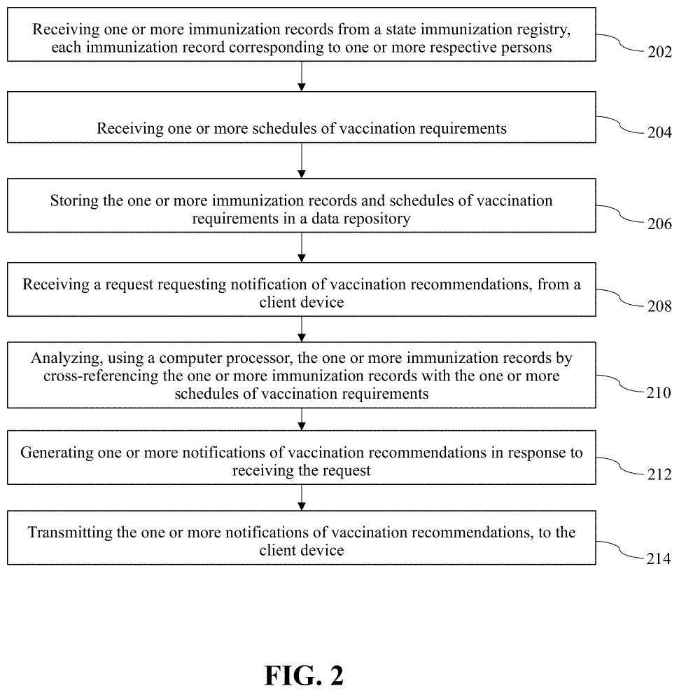

FIG. 2 illustrates a computer implemented method for tracking immunization, in accordance with embodiments of the invention disclosed herein. The method begins at step 202 when the processor 110 receives at least a portion of one or more immunization records 123 from a state immunization registry (as a third party device), with each immunization record 123 corresponding to one or more respective persons (e.g., 103, 120). At step 204, the processor 110 receives one or more schedules of vaccination requirements 113. At step 206, the processor 110 stores the one or more immunization records 123 and schedules of vaccination requirements 113 in a data repository 112, so that the one or more records 123 may be accessed in the future by any device connected with the network 104. Further, the data repository 112 has a plurality of data cells to store one or more records 123 in dedicated data cell corresponding to one or more respective family members 120. At step 208, the processor 110 receives a request requesting notification of vaccination recommendations 126, from a client device 102. At step 210 the processor 110 analyzes, using a computer processor, the one or more immunization records 123 by cross-referencing the one or more immunization records 123 with the one or more schedules of vaccination requirements 113. At step 212 the processor 110 generates one or more notifications of vaccination recommendations 126 in response to receiving the request. At step 214 the processor 110 transmits the one or more notifications of vaccination recommendations 126, to the client device 102.

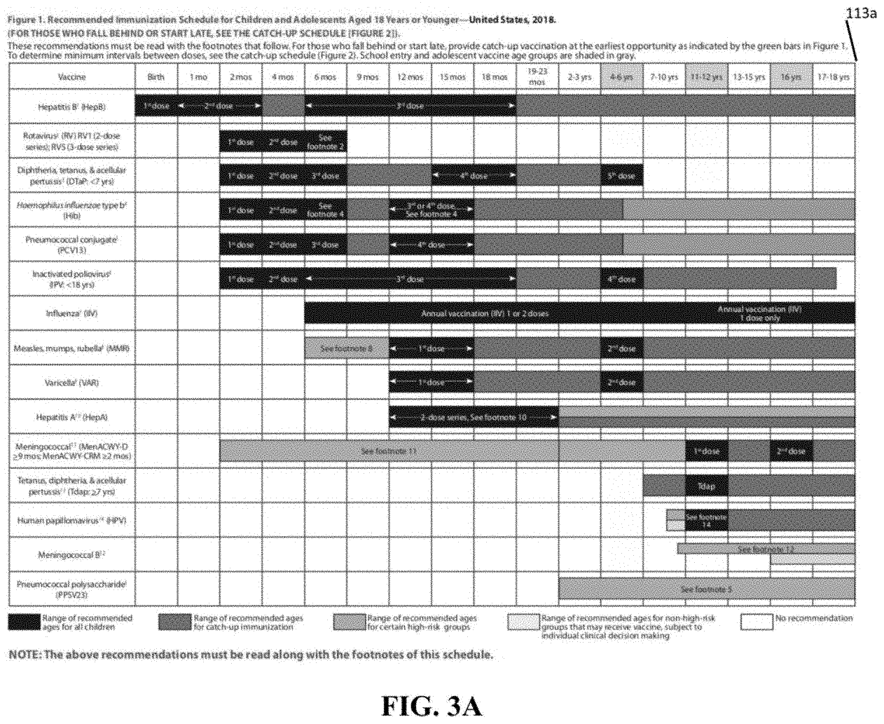

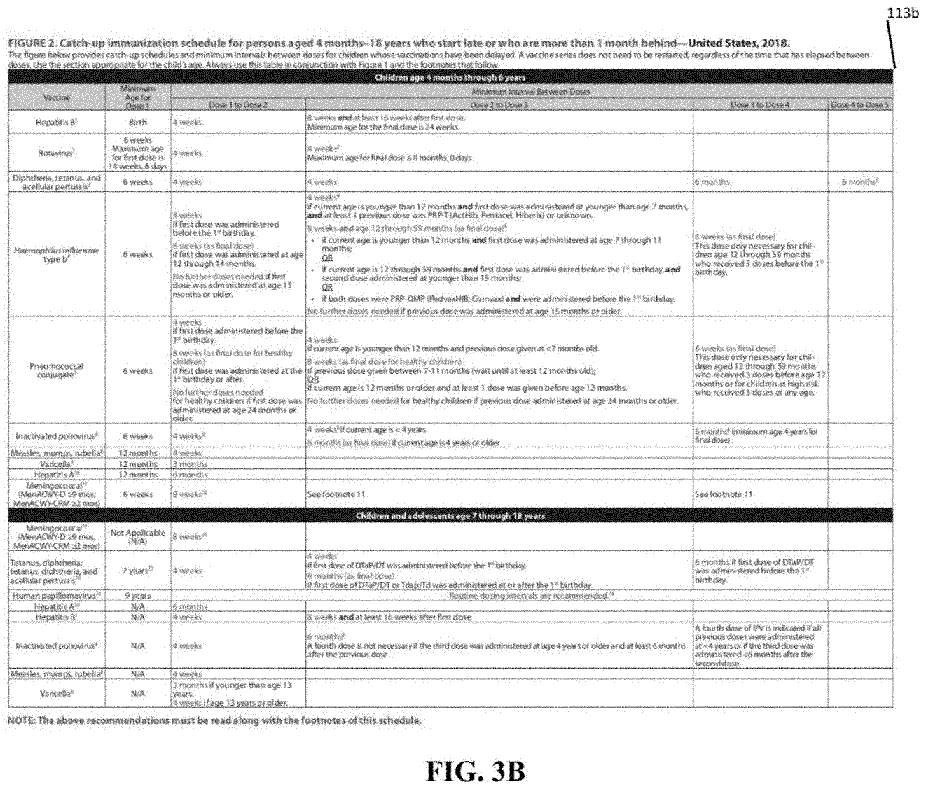

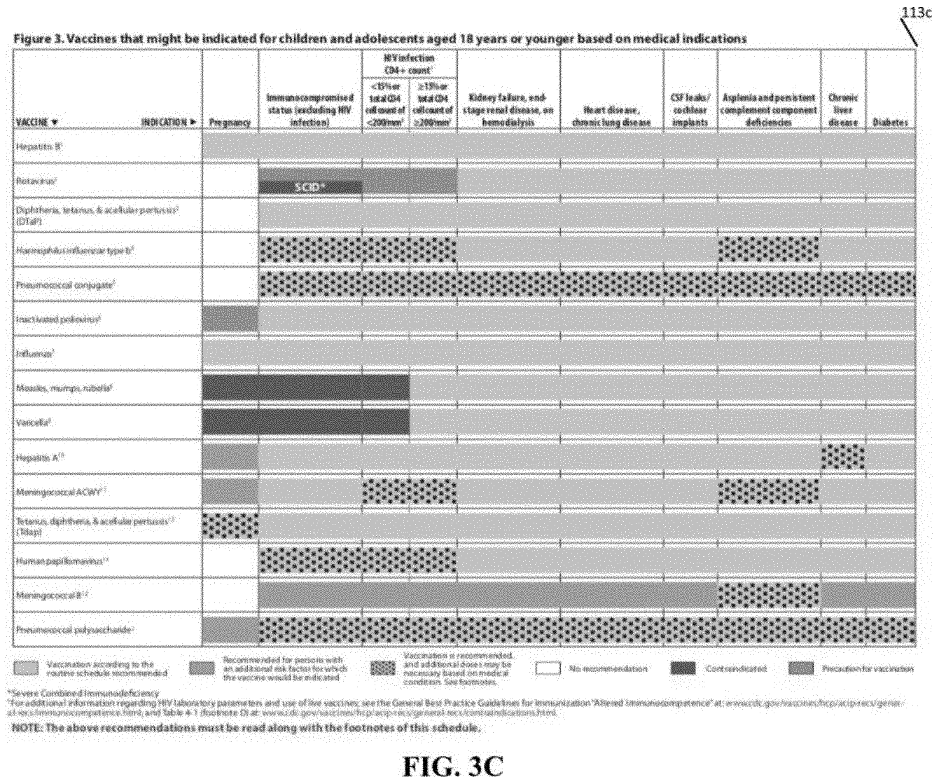

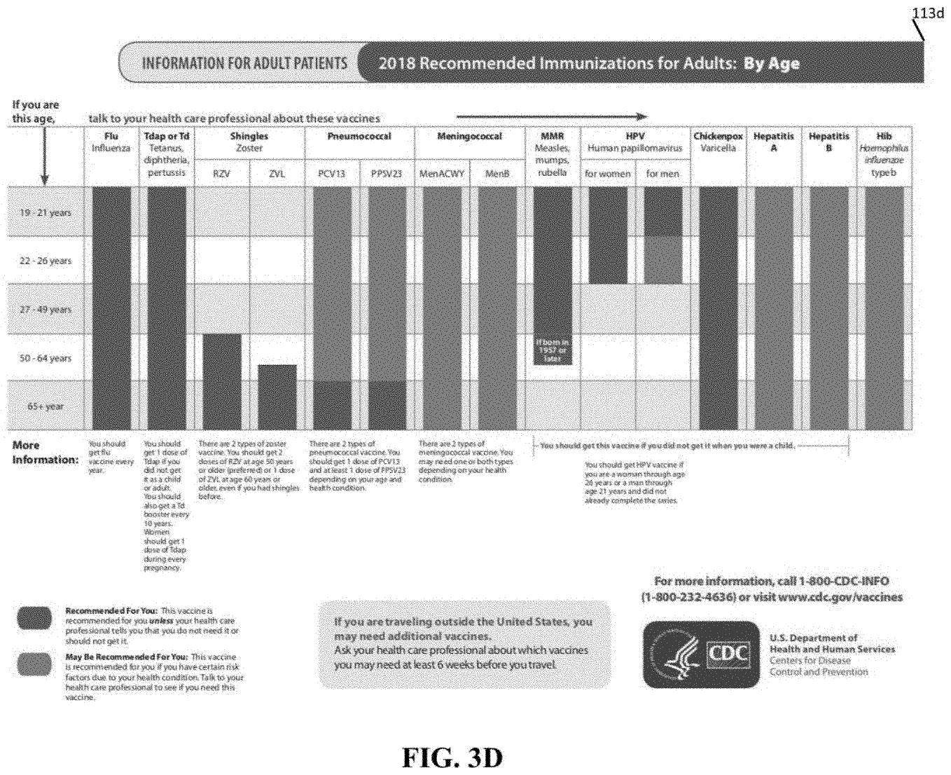

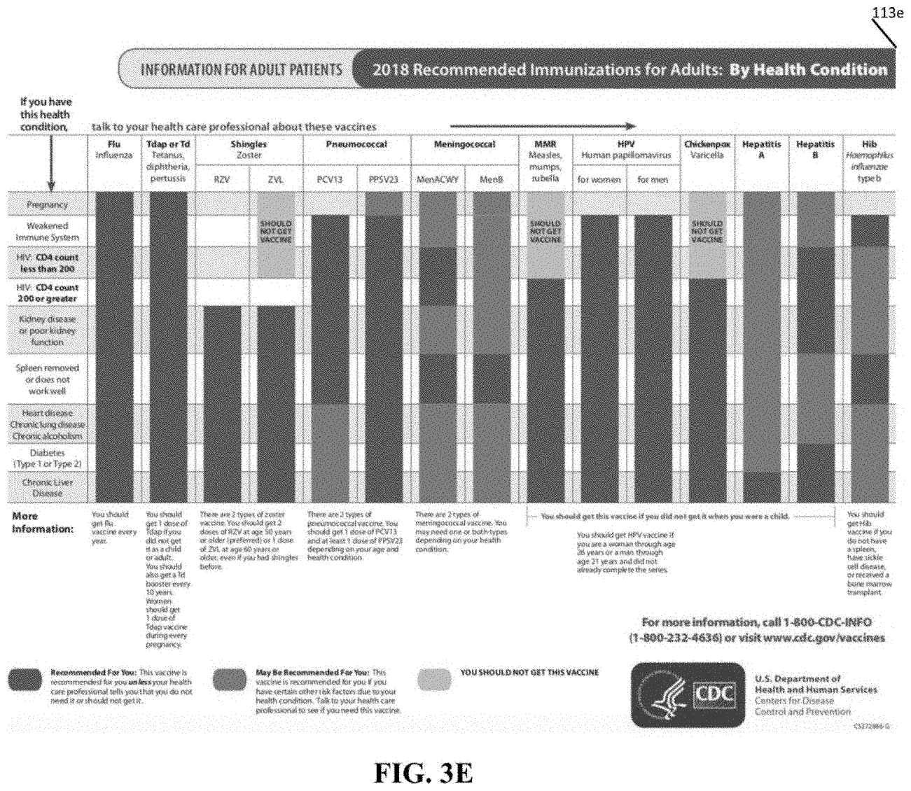

In a preferred embodiment, the processor 110 receives one or more immunization records 123 from a State immunization registry. The client device 102 is preferably in networked 104 communication with the computer system 106. Preferably, each immunization record of the one or more immunization records includes a name, a date of birth (DOB), a gender, a birth state, medical history and allergies as shown in FIG. 3.

In yet another preferred embodiment, the processor 110 receives one or more immunization records 122, 123 corresponding to one or more respective family members, from the client device 102. The client device 102 is preferably in networked 104 communication with the computer system 106. The one or more family members 120 may or may not be in blood relation to each other. The one or more respective family members 120 may include a father, a mother, sons, daughters, cousins and friends. Each immunization record 122, 123 of the one or more immunization records of the family members includes a name, a date of birth (DOB), a gender, a birth state, medical history and allergies of the family member(s) 120.

At step 210, the processor 110 analyses the one or more immunization records 122 and generates one or more recommendations 126 based on the one or more immunization records 122. Further, in a preferred embodiment, the one or more recommendations 126 are selected from a group consisting of, for example, medicine, therapy, counseling and vaccination. At step 208, the processor 110 transmits the one or more recommendations 126 to the client device 102. Furthermore, the processor 110 notifies the client device 102 for vaccination 126 corresponding to the one or more respective family members 120. The processor 110 then notifies the client device 102 for upcoming vaccinations 126 corresponding to the one or more respective family members. Also, the processor 110 notifies the client device 102 for due vaccinations 126 corresponding to the one or more respective family members 120. Additionally, the processor 110 notifies the client device 102 for recommended vaccinations 126 based on one or more dates of birth (DOB), corresponding to the one or more respective family members 120.

In accordance with preferred embodiments disclosed herein, the processor 110 further provides a schedule for vaccinations 113 corresponding to the one or more family members 120, where the schedule for vaccinations being received from the third party device 114. Preferably, the schedule for vaccination 113 is designed to include schedules for one or more age groups. Further, the one or more age groups are preferably selected from a group consisting of, for example, 0-6 years, 7-18 years, adults by age and adults by health condition. Referring to FIG. 3A, that illustrates a snapshot of an exemplary schedule for vaccination 113 for 0-6 years of age group, in accordance with an embodiment of the invention. Preferably, the one or more recommendations 126 for general vaccination schedule is generated wherein a vaccine name and a respective month is provided. Further, the vaccine name may be, for example, HepatitisB, Rotavirus (RV) and Diphtheria, Tetanus, or Pertussis (DTaP). A shot of Hepatitis B vaccine is safe and effective and is usually given as 3-4 shots over a six month period. A Rotavirus (RV) vaccine is given in 3 doses at ages of 2 months, 4 months, and 6 months to protect a child against rotavirus illness. Rotavirus (RV) is a virus that spreads easily among infants and young children. The virus causes severe watery diarrhea, vomiting, fever, and abdominal pain. Further, a DTaP vaccine is given at 2, 4, and 6 months of age, with the fourth dose given at the age between 15 to 18 months and the fifth at the age between 4 to 6 years to protect the child against three diseases: diphtheria, tetanus, and pertussis (whooping cough). Preferably, the one or more recommendations 126 are provided for the age group of 0-6 years. For example, two doses given at least four weeks apart for children aged 6 months through 8 years of age that are getting an influenza (flu) vaccine for the first time and for some other children of similar age group and two doses of Hepatitis A vaccine are required for lasting protection. The first dose of Hepatitis A vaccine is given between 12 months and 23 months of age. The second dose is given 6-18 months later. In case the child (e.g., 120) is suffering from an allergy (e.g., 130), or the child has a risk associated medical condition that puts the child at risk for infection, the one or more additional vaccines are recommended 126 to the child for treatment.

Further referring to FIG. 3A, that illustrates a snapshot of an exemplary recommended immunization schedule 113a for children and adolescents aged 18 years or younger, in accordance with embodiments of the invention. As shown in FIG. 3A, the exemplary one or more vaccines based on age include Human Papillomavirus (HPV), Meningococcal Conjugate Vaccine (MCV), Pneumococcal Conjugate Vaccine (PCV13) or Pneumococcal Polysaccharide Vaccine (PPSV23), Hepatitis A series, Hepatitis B series, Inactivated Polio Vaccine (IPV) series, Measles, Mumps, Rubella (MMR) series, Varicella Vaccine series and Diphtheria, Tetanus, Pertussis (DTaP). Human Papillomavirus (HPV) vaccine is given at age 11-12 to protect against HPV related disease, Meningococcal Conjugate Vaccine (MCV) is recommended at age 11-12 years. Additionally, a booster shot is of a vaccine is required periodically to ‘boost’ the immune system. For example, a booster shot of the tetanus and diphtheria (Td) vaccine is recommended 126 for adults every 10 years. Children who receive MCV for the first time at age 13-15 years gets one-time booster dose between 16-18 years of age and Pneumococcal Conjugate Vaccine (PCV13) and Pneumococcal Polysaccharide Vaccine (PPSV23) are recommended for children at 6-18 years of age with high risk medical conditions.

Referring to FIG. 3B, that illustrates a snapshot of a recommended catch-up immunization schedule 113b for persons aged 4 months-18 years who start late or who are more than 1 month behind the recommended immunization schedule illustrated in FIG. 3A for children and adolescents aged 18 years or younger, in accordance with embodiments of the invention.

Referring to FIG. 3C, that illustrates an exemplary recommended immunization schedule 113c of recommended vaccines that might be indicated for children and adolescents aged 18 years or younger based on medical indications, in accordance with embodiments of the invention.

Referring to FIG. 3D, that illustrates an exemplary recommended immunization schedule 113d for adults by age, in accordance with embodiments of the invention. Further, one or more recommended vaccines are provided based on 19 to 65+ years of age. Subsequently, one may consult a health care professional before receiving the one or more vaccines based on, but not limited to, life-style, age health and job.

Referring to FIG. 3E, that illustrates an exemplary recommended immunization schedule 113e for adults by health condition, in accordance with embodiments of the invention. As shown in FIG. 3E, in one embodiment, the exemplary recommended immunization schedule 113e is provided on the basis of health conditions such as, preferably, pregnancy, weakened immune system, (Human immunodeficiency Virus) HIV, kidney disease or poor kidney function, Asplenia, heart disease, chronic lung disease, chronic alcoholism, diabetes (Type 1 or Type 2), and chronic liver disease.

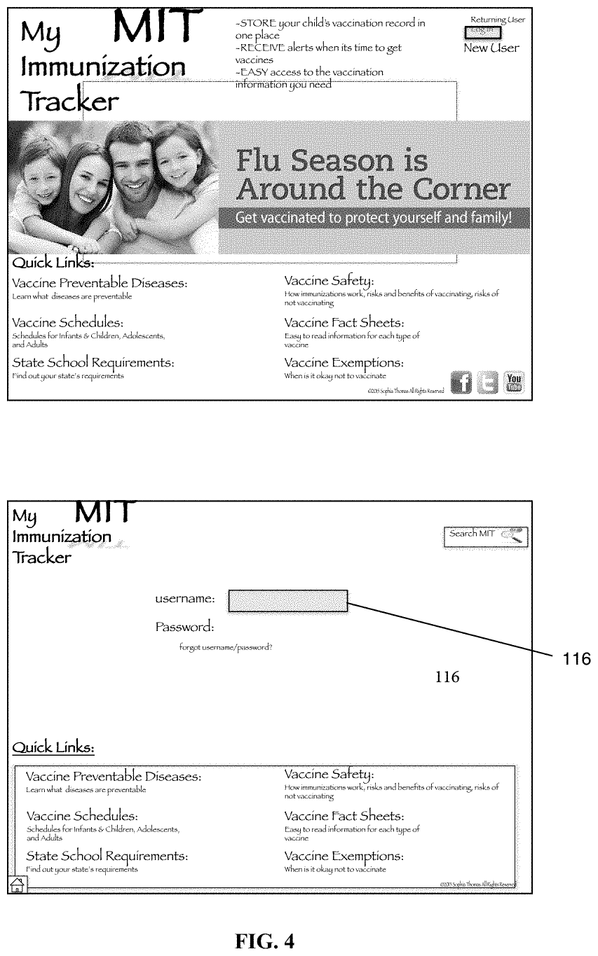

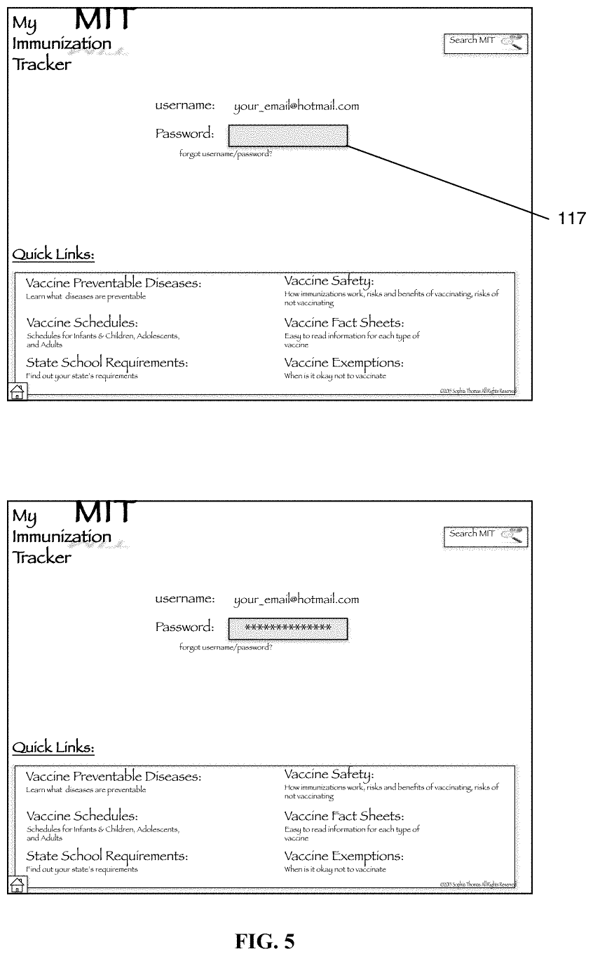

FIGS. 4 and 5 show exemplary diagrams of windows of embodiments of an immunization tracker application of the system. Username 116 and password 117 prompts request login credentials from a user 103. Username 116 maybe be an email address, for example.

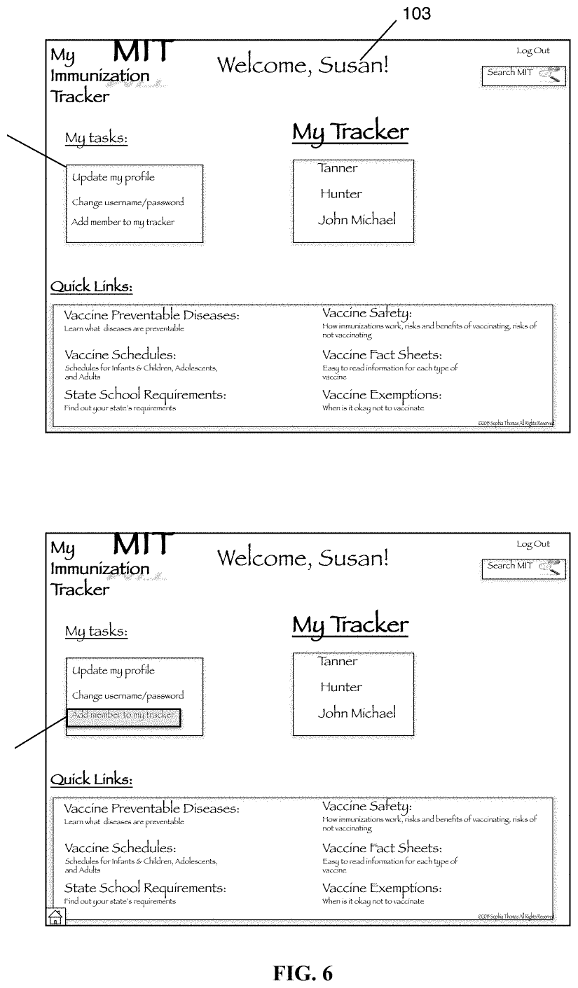

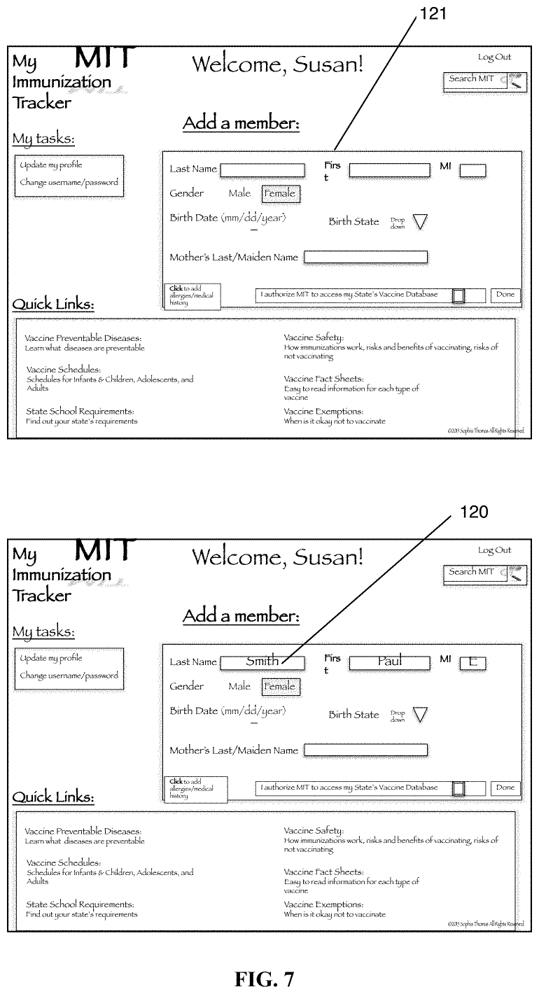

FIGS. 6-8 show exemplary diagrams of windows of embodiments of an immunization tracker application of the system. In a preferred embodiment, a prompt 118 to add a family member 120 solicits input 121 from a user 103 to provide a family member's name, gender, birth date, birth state, and mother's last name or maiden name. Preferably, the application is configured to solicit any information deemed relevant or useful to include into a user's immunization record 122 of the family member 120.

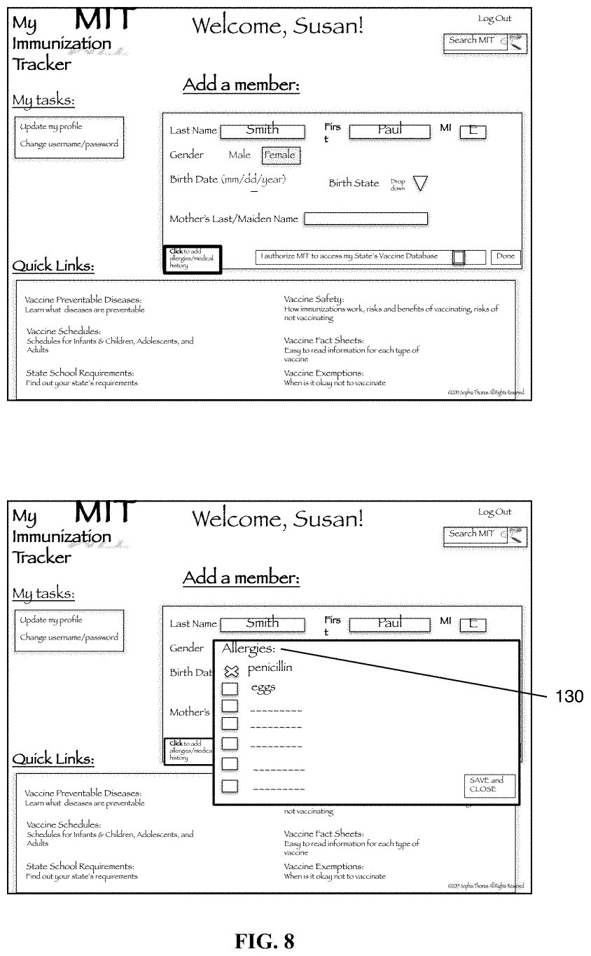

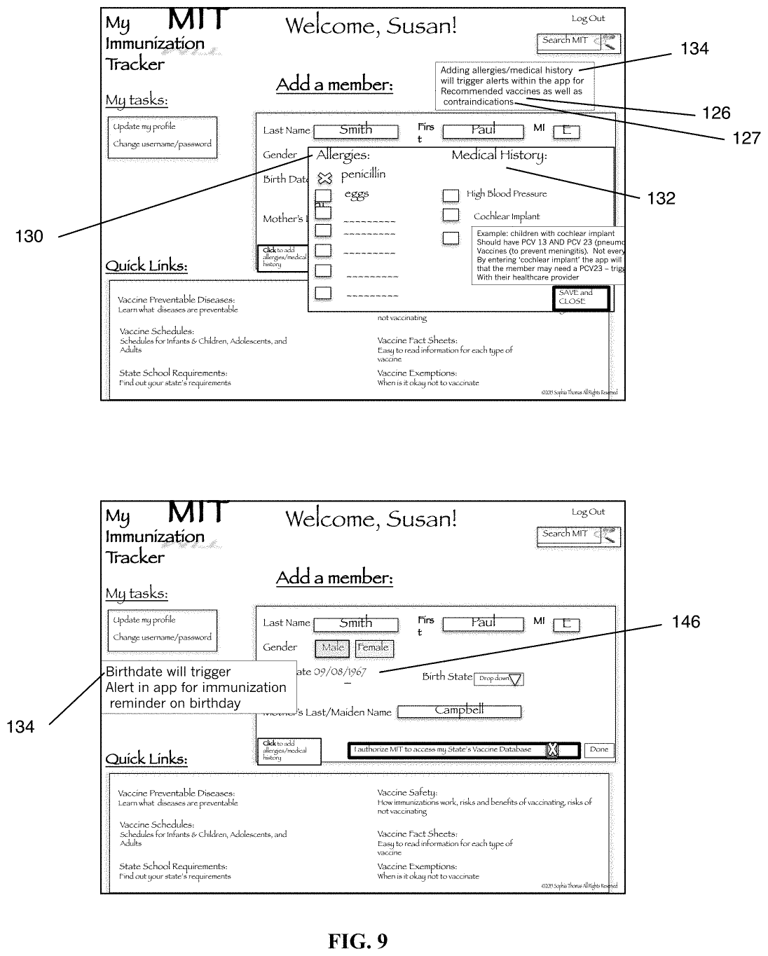

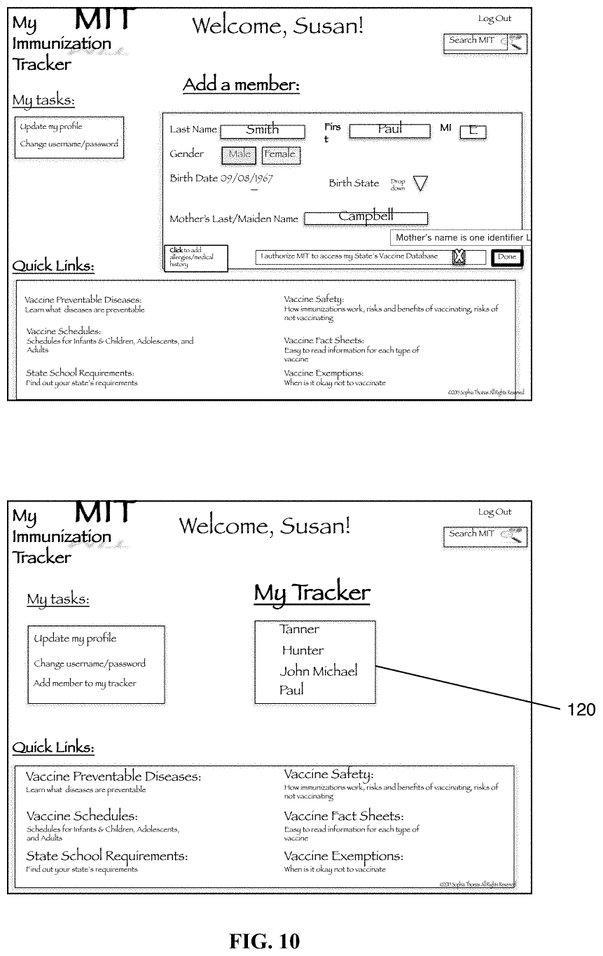

Referring to FIGS. 8 and 9, by adding allergies 130 and medical history 132 into the application, the application will trigger alerts 134 within the application for recommended vaccines 126 as well as contraindications 127. For one example of a recommended vaccine 126, children with cochlear implants should have PCV13 and PCV23 (pneumococcal) vaccines (to prevent meningitis). However, not everyone needs PCV23. By a user 103 entering ‘cochlear implant’ into the application, the application will alert 134 the user 103 that the user 103 (or family member 120) may need a PCV23, which will trigger a conversation with the family member's 120 healthcare provider. By entering a birthdate 146 of a user or of a user's family member 120, the application will trigger an alert 134 in the application for an immunization reminder on the family member's birthday 146. FIG. 10 shows an exemplary diagram of a window of an embodiment of an immunization tracker application of the system listing family members 120 after a new member has been added.

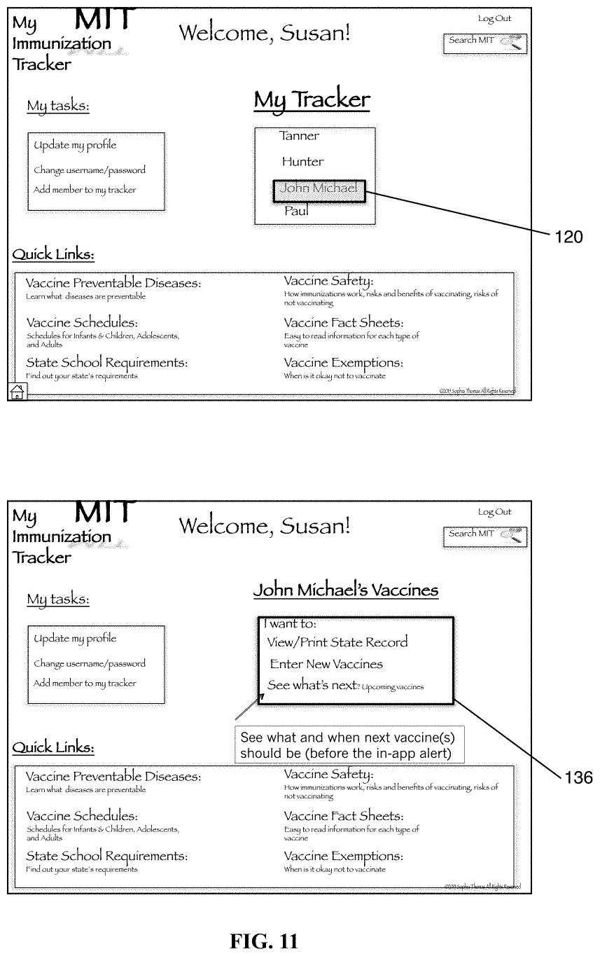

Referring to FIG. 11, an exemplary diagram of a window of an embodiment of an immunization tracker application shows an exemplary family member 120 selected and a screen 136 indicating which vaccines should be scheduled next. This allows the user to view the vaccine schedule at a time before an in-app alert 134.

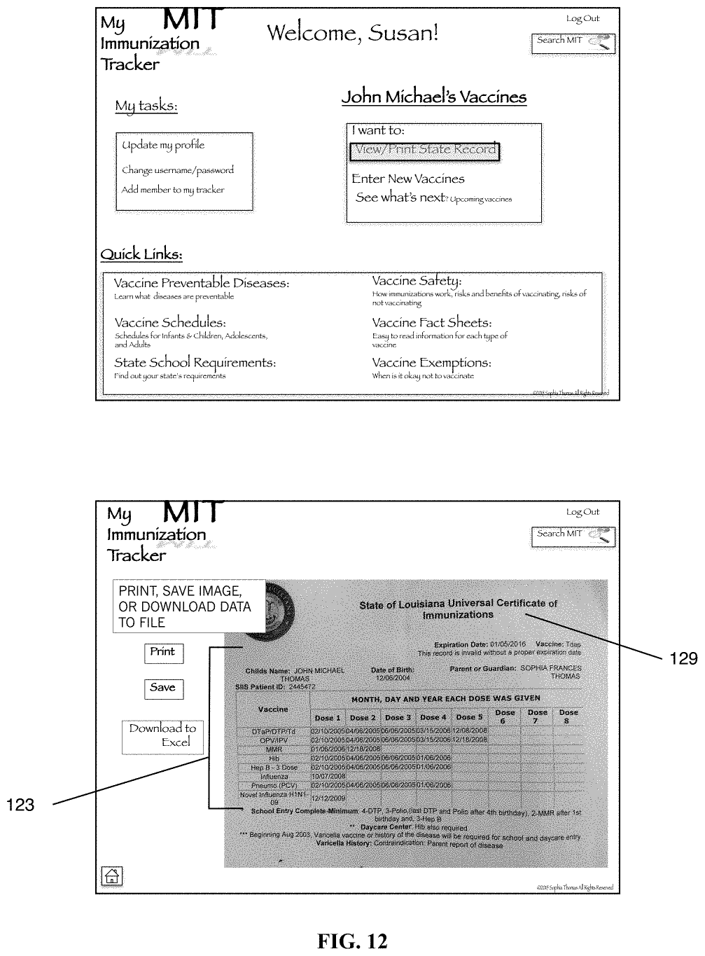

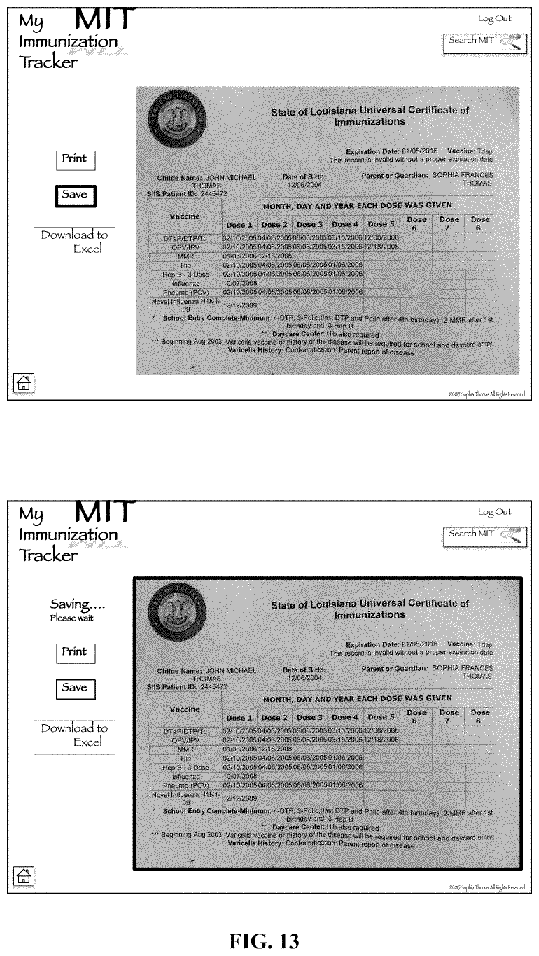

Referring to FIGS. 12 and 13, exemplary diagrams of a window of an embodiment of an immunization tracker application of the system shows a family member 120 selected and an option to view/print a State immunization record 123. A State immunization record 123, such as a certificate of immunizations 129, is preferably stored within the application. Other medical records are preferably stored within the application for ease of access by a user 103. Preferably, options to print, to save the image, or to download the data to a file are provided.

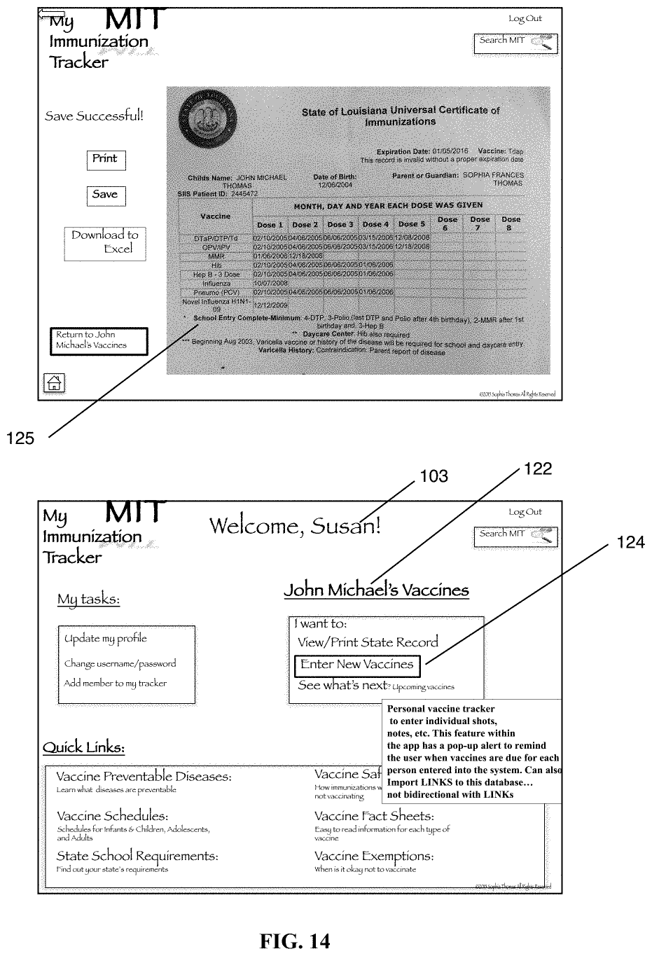

Referring to FIGS. 14-16, exemplary diagrams of exemplary windows of embodiments of an immunization tracker application of the system show an exemplary procedure for a user 103 to enter new vaccines and notes into the application. For example, each family member 120 preferably has a personal vaccine tracker 124 to enter individual shots 148, notes, and other relevant information. This personal vaccine tracker 124 within the application preferably includes a pop-up alert 134 to remind the user 103 when vaccines are due for each family member 120 entered into the system. In certain embodiments, immunization records 123 from governmental or other centralized record keeping entities, such as Louisiana Immunization Network for Kids Statewide (LINKS), are preferably imported into the application. In a preferred embodiment, functionality to make the application bidirectional is provided, such that the individual shot records 148 entered by a user 103 and synced with the immunization records 123 of the governmental or other centralized record keeping entities, providing synchronized immunization records 122 information on the client device 102 and the third party device 114.

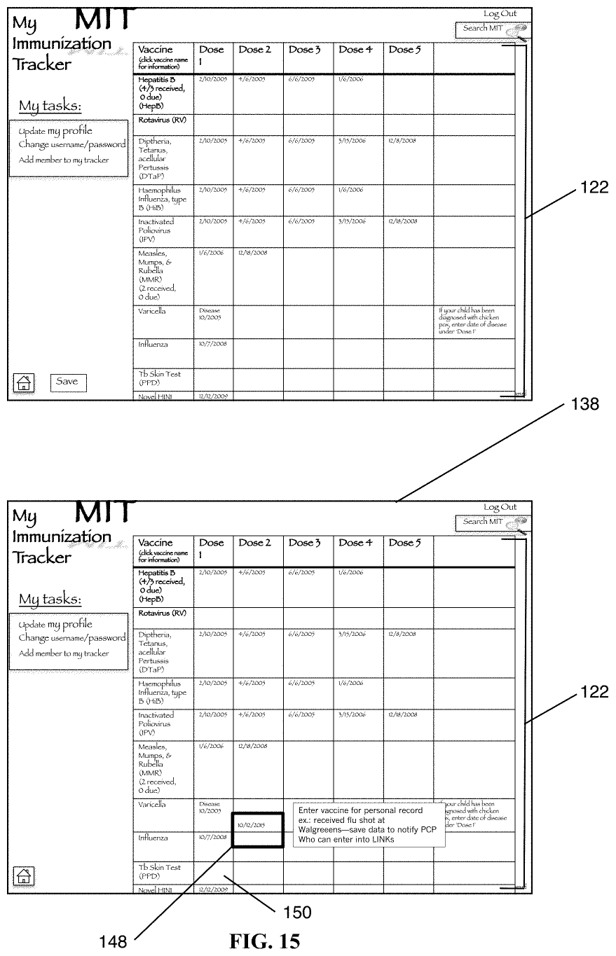

Referring to FIG. 15, snapshots of exemplary windows 138 of an immunization tracker application is provided, illustrating a user 103, such as a family member 120, entering details of individual vaccine shots 148 of which one of the user's family members 120 (e.g., John Michael) has already received, in accordance with embodiments of the invention. Referring to FIGS. 14-15, exemplary user 103 “Susan” enters an individual vaccine 148 into a personal immunization record 122 of exemplary family member 120 “John Michael”, such as, after a flu shot is received by John Michael, such as at a pharmacy such as Walgreens. In one embodiment, the system notifies John Michael's primary care physician (“PCP”), who can enter the individual vaccine 148 data into a governmental data network, such as LINKS. Herein, the processor receives details of the one or more vaccines 148 into one or more respective records 122, from the client device 102. Further, the processor 110 keeps track of the details of vaccination 148 received by the one or more family members 120 (e.g., John Michael) and stores the details of the vaccinations in the data repository 112. Further, the processor 110 notifies the client device 102 for upcoming vaccinations. In a preferred embodiment, the system provides pop-up notification alerts for outbreaks, as well as pop-up reminder alerts (e.g., 134) on a client device 102, such as, for example, “Flu season has arrived, time to get your flu shot” or “Happy 4th birthday to Susan. Don't forget to schedule her 4-year checkup and immunizations” or “Susan's birthday was 2 weeks ago, did you schedule her immunizations?” In addition, through embodiments of the present invention, an individualized immunization forecast is provided for upcoming immunizations based upon the information in the user's immunization record 122. In a preferred embodiment, the method analyzes, using a computer processor 110, the one or more immunization records 122 by cross-referencing the one or more immunization records 122 with the one or more schedules of vaccination requirements 113. The processor 110 then notifies the client device 102 for recommended vaccinations that have not been received (e.g., at 150) by the user 103 or the family member 102 in the past.

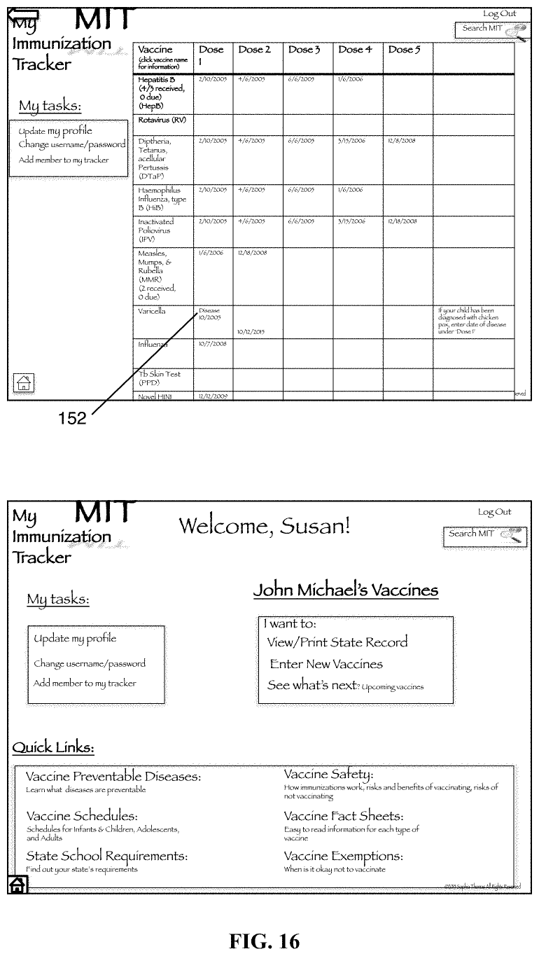

Referring to FIG. 16, exemplary user 103 “Susan” also enters a date of a disease 152, such as chicken pox, into a personal immunization record 122 of an exemplary family member 120, such as, “10/2005” at 152 for “Varicella” in FIG. 16. Herein, the processor receives details of the one or more date of a disease 152 into one or more respective records 122, from the client device 102. Further, the processor 110 keeps track of the details of the date of the disease 150 of the one or more family members 120 and stores the details of the date of the disease 150 in the data repository 112. Further, the processor 110 notifies the client device 102 for upcoming vaccinations recommendations.

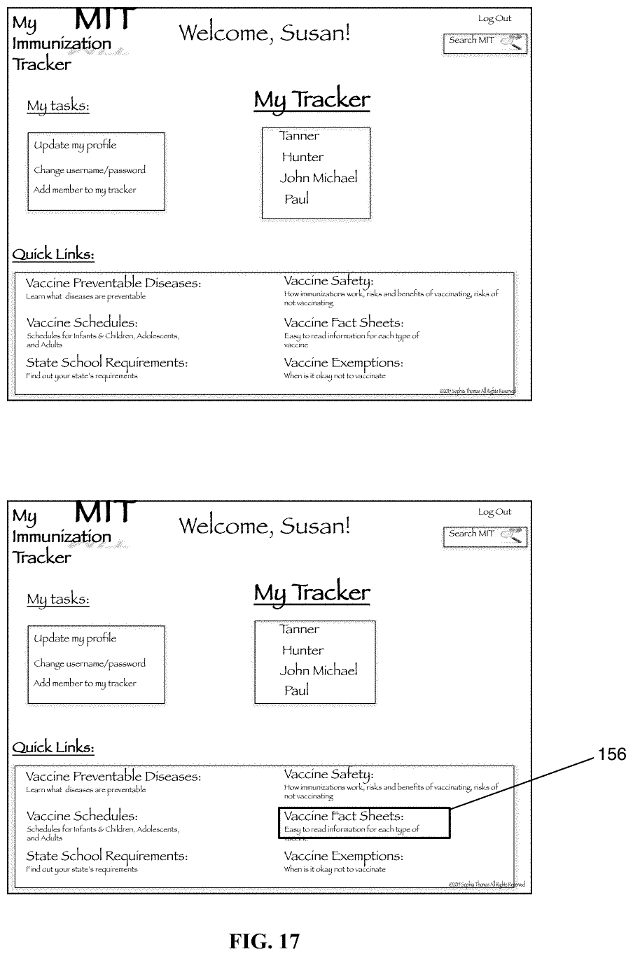

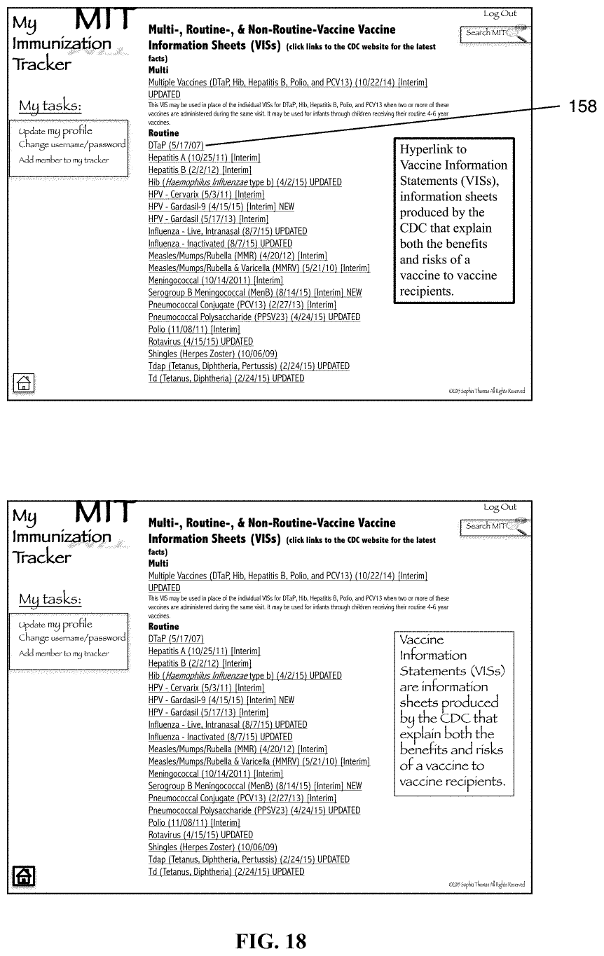

Referring to FIGS. 16-18, in a preferred embodiment, the processor 110 transmits to the client device 102 vaccine facts information 154, received from the third party device 114, such as from CDC, related to the one or more vaccination recommendations. In one embodiment, the user 103 clicks on Vaccine Facts Sheets 156 and receives vaccine facts information 154. Preferably, the vaccine facts information 154 preferably includes risks and benefits related to the one or more recommendations. For example, where one or more vaccinations are provided as the one or more recommendations corresponding to the one or more family members, the processor 110 preferably transmits the vaccine facts information 154 in a form of one or more weblinks 158, which provide webpages explaining the risks and benefits related to the one or more vaccinations. Preferably, the computer system 106 receives the vaccine facts information 154 and transmits that information to the client device 102. For example, there is provided a hyperlink to Vaccine Information Statements (VISs) information sheets produced by the CDC to explain both the benefits and risks of a vaccine to vaccine recipients.

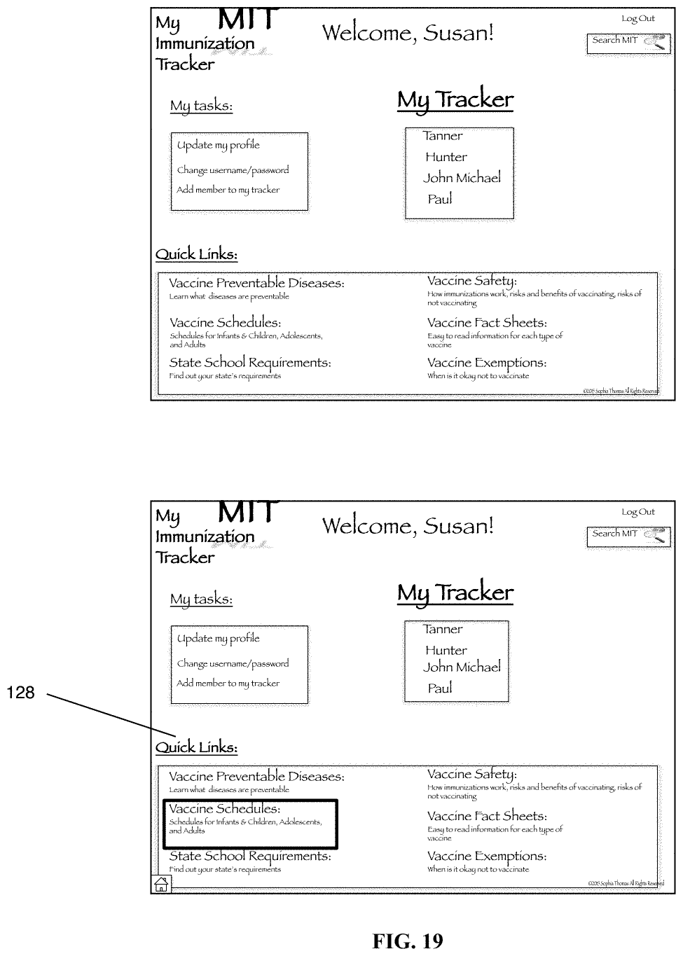

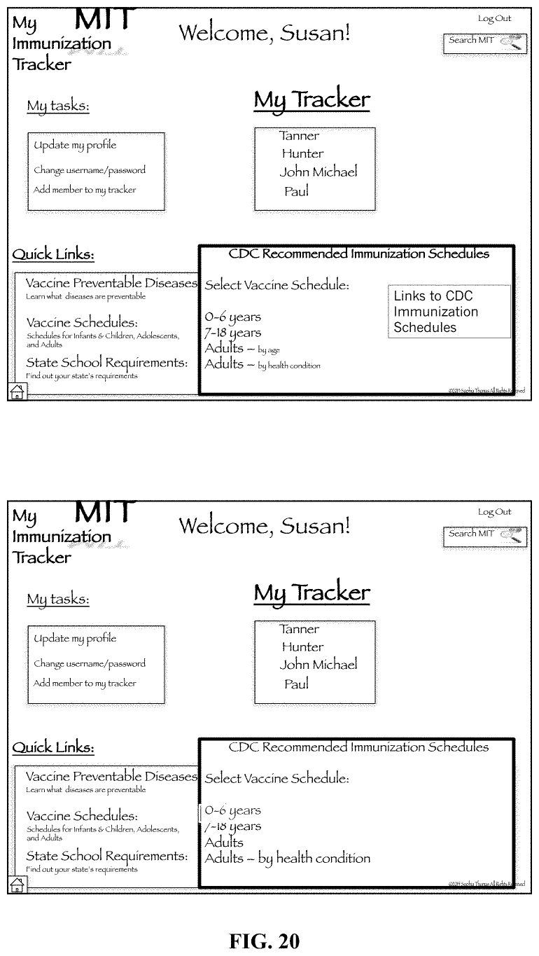

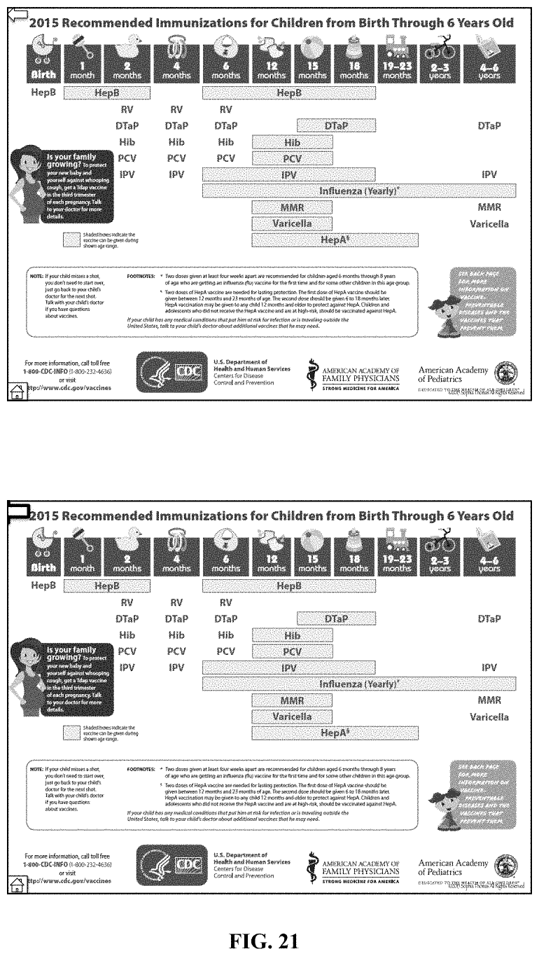

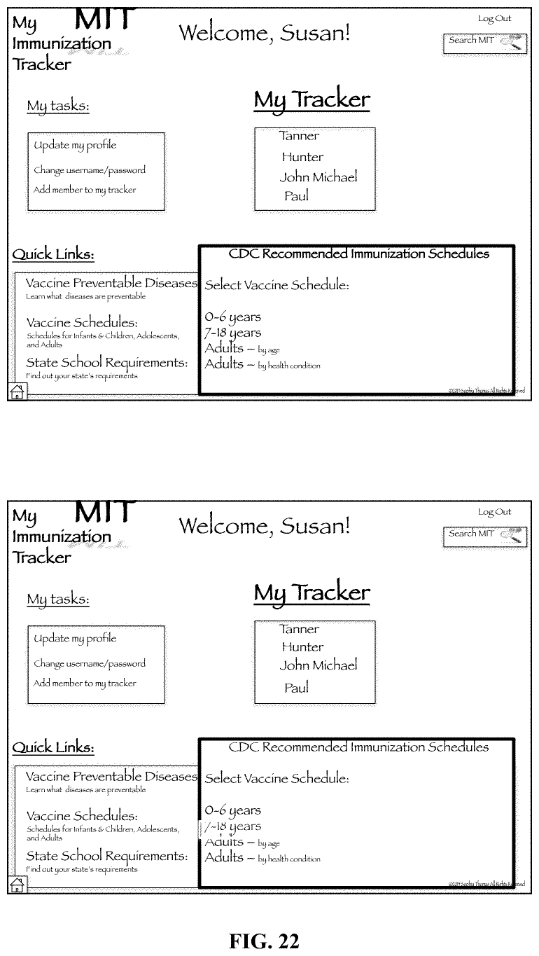

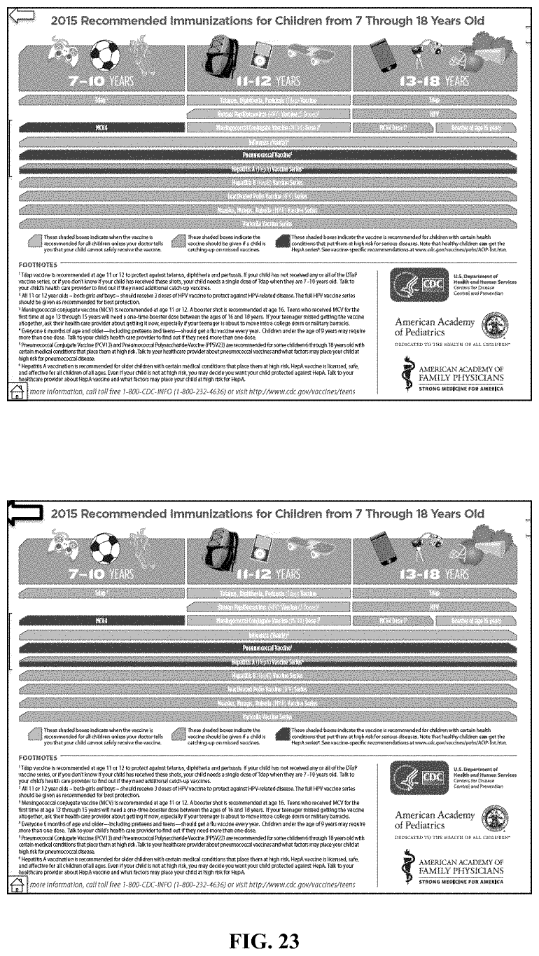

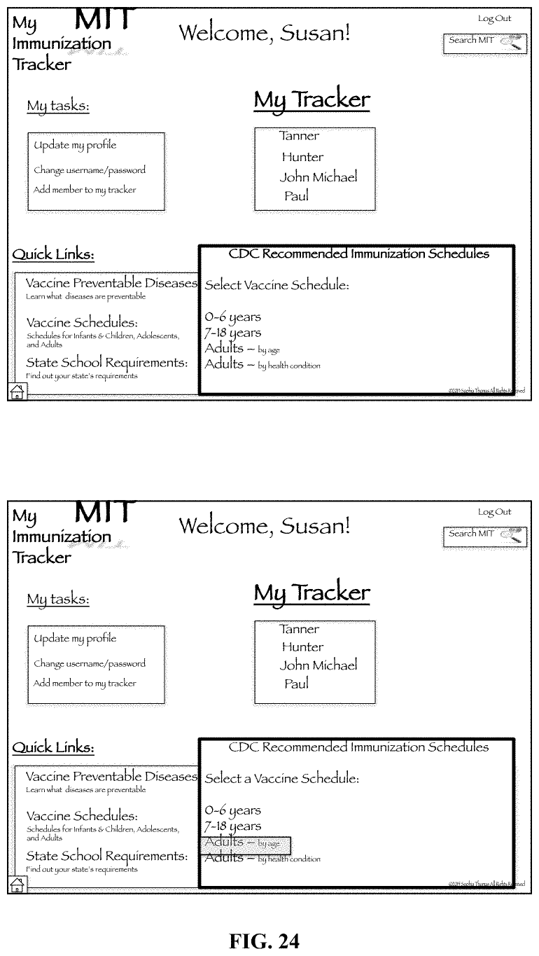

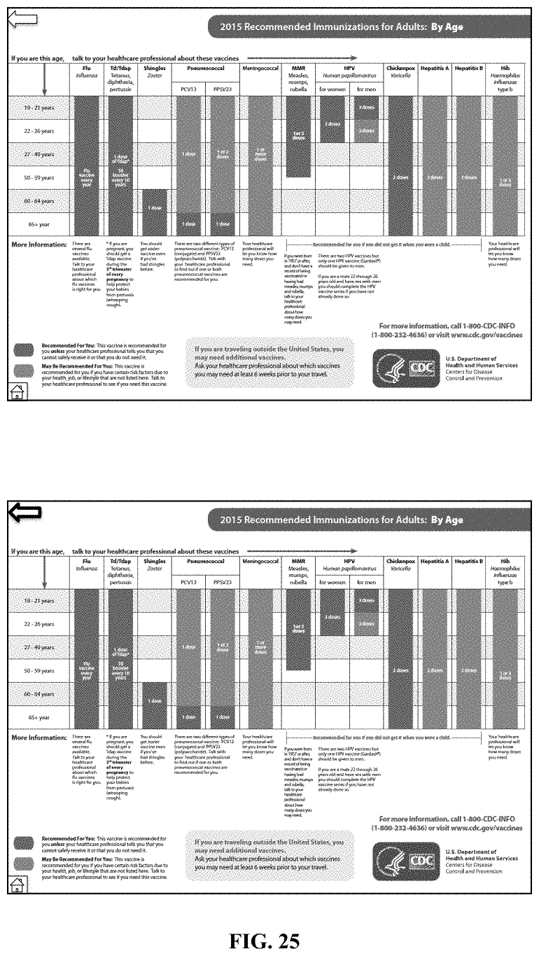

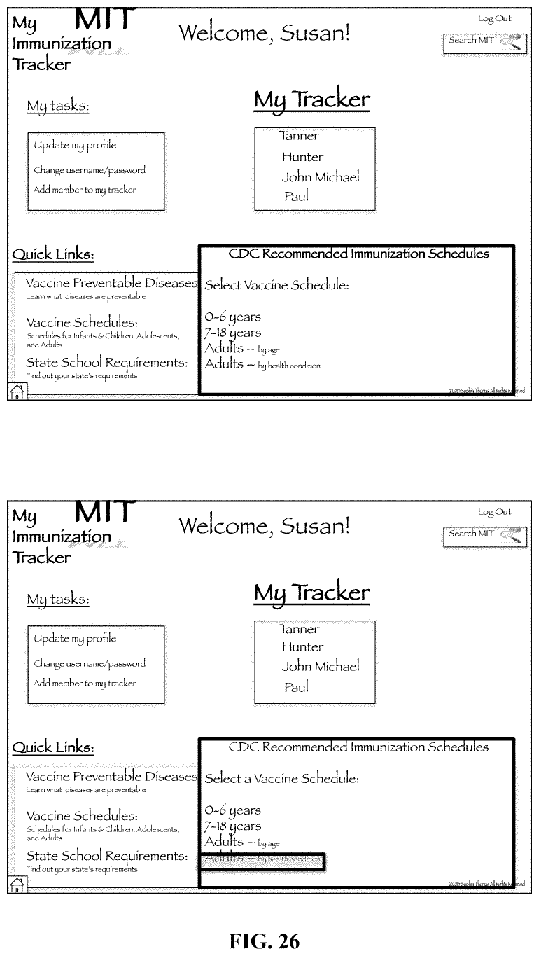

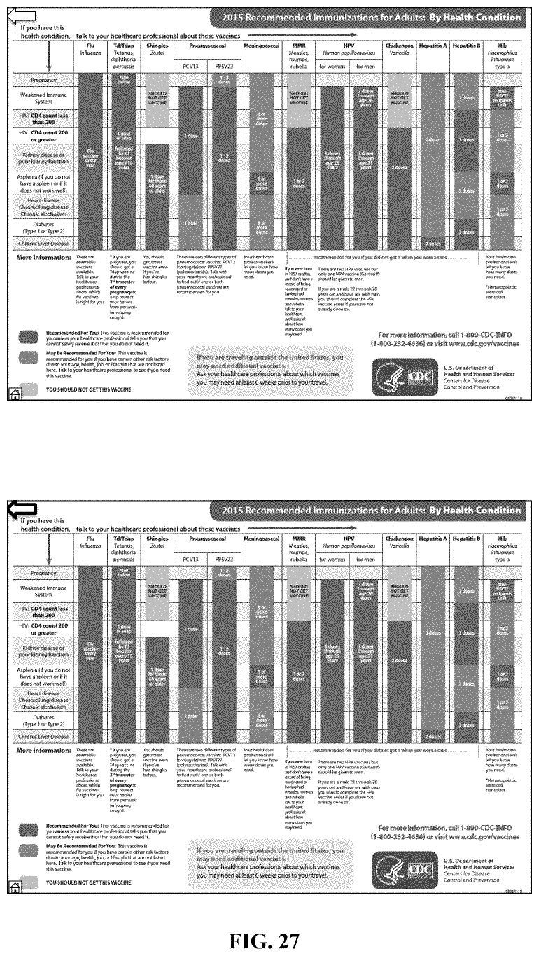

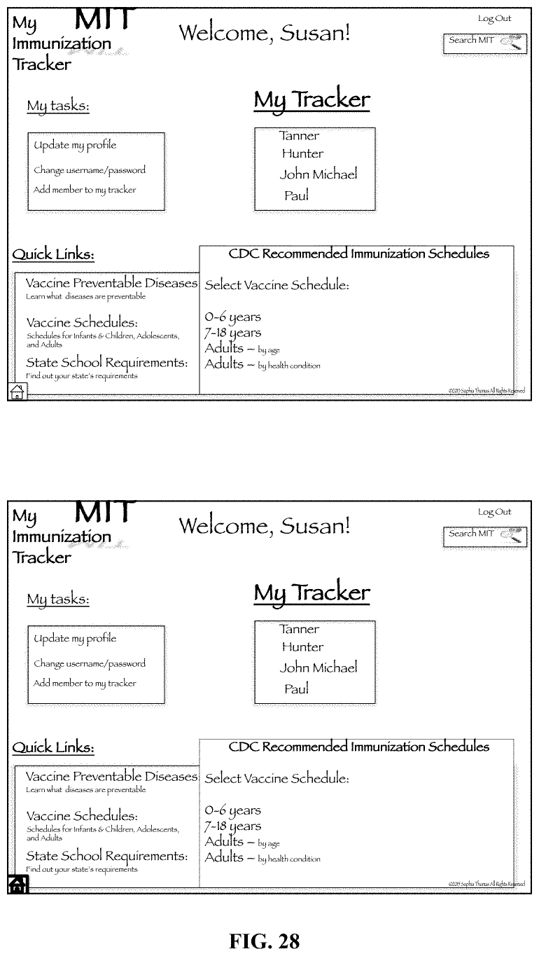

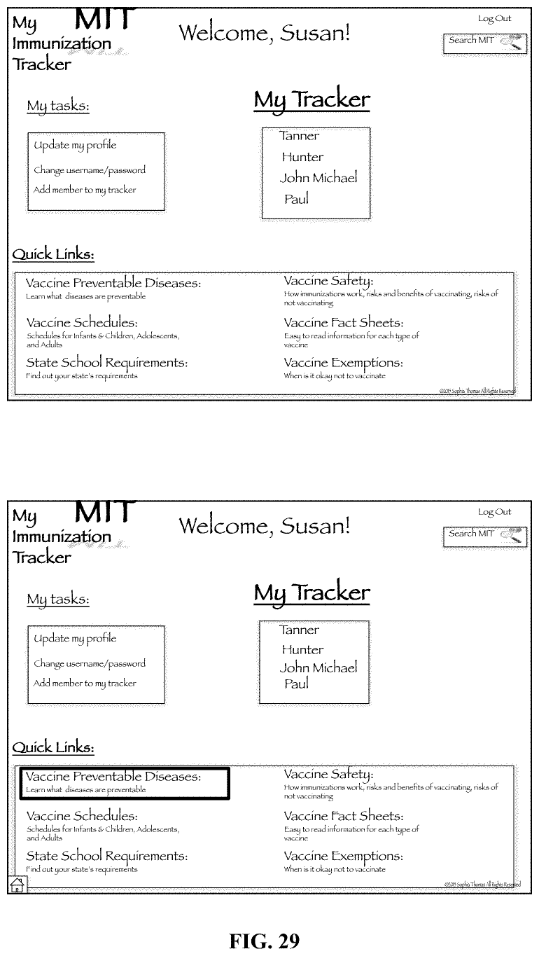

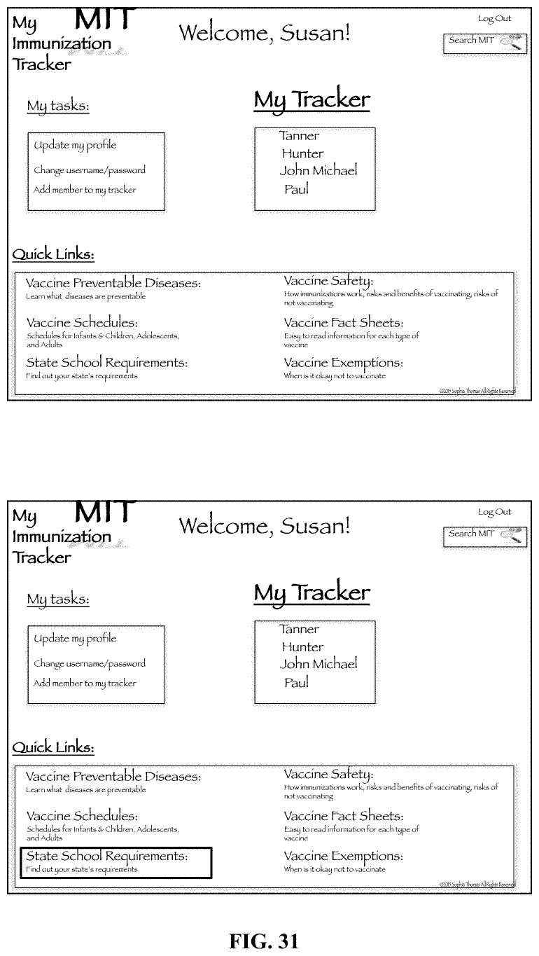

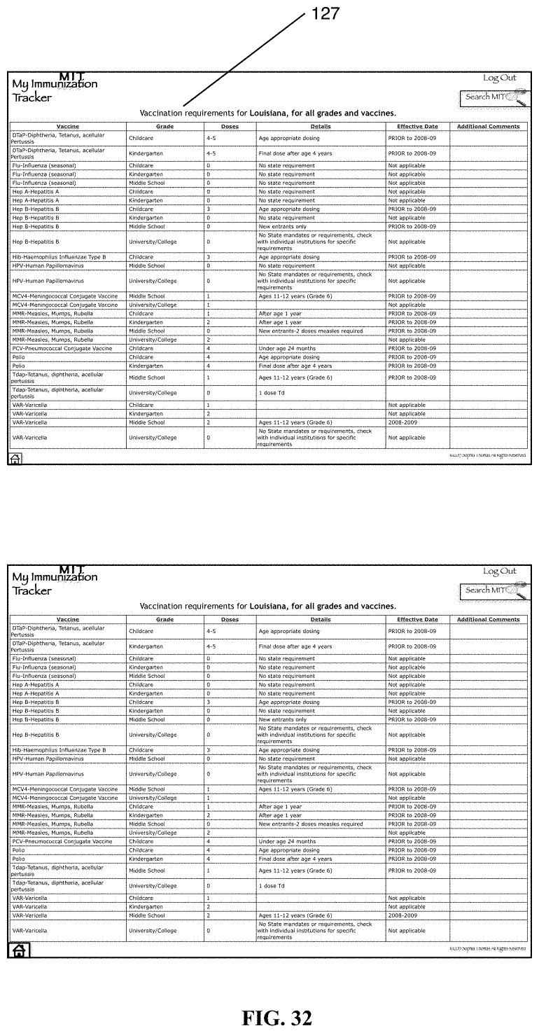

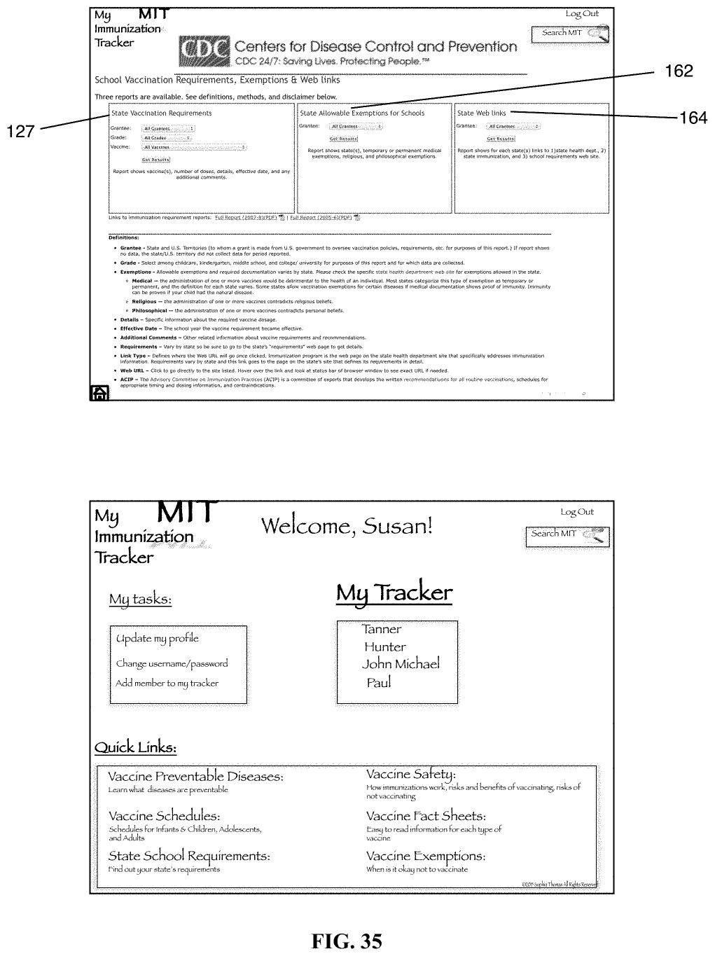

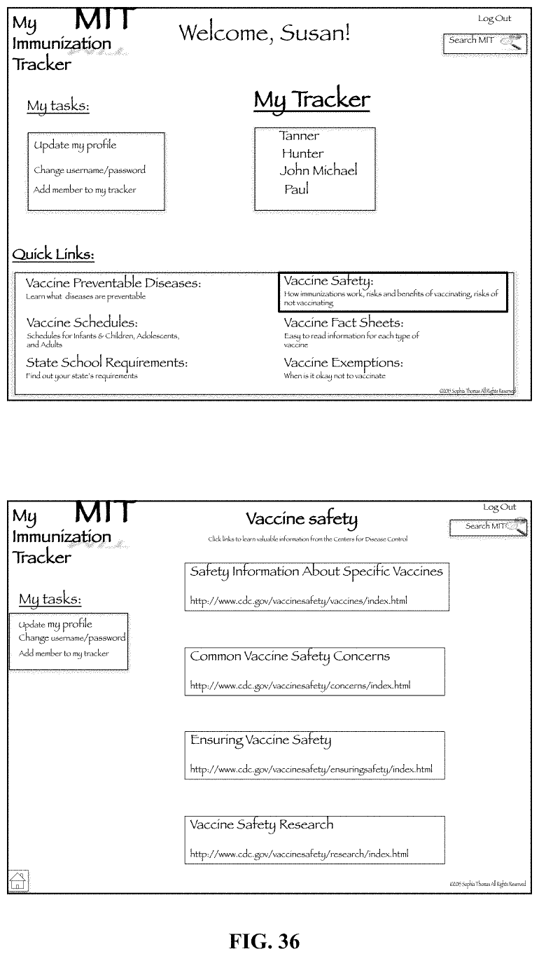

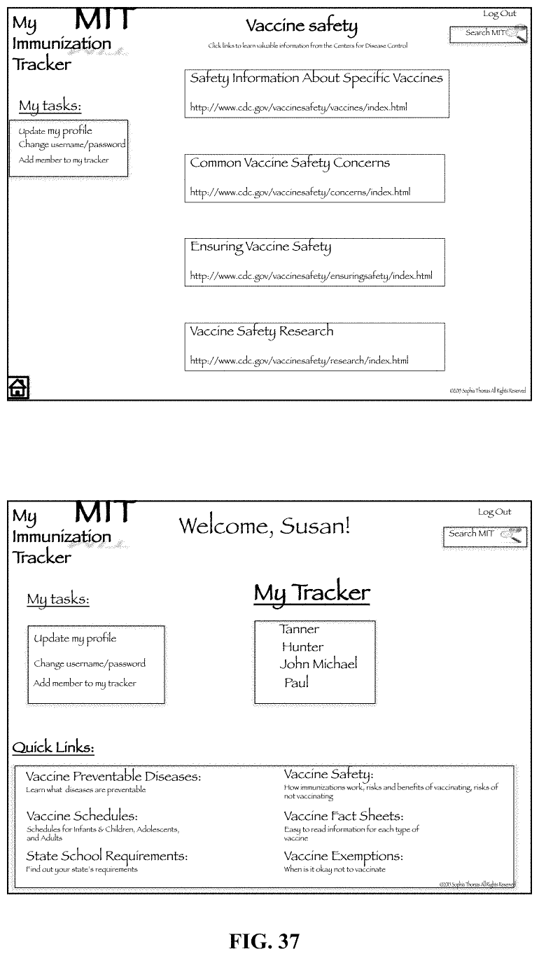

Referring to FIGS. 19-37, exemplary diagrams of windows of an embodiment of an immunization tracker application of the system show an exemplary set of quick links 128 in a portion of the window. Quick links 128 may include links to information on vaccine preventable diseases, vaccine schedules, State requirements 127 (e.g., in FIG. 32), school requirements 125, vaccine safety, vaccine fact sheets, and vaccine exemptions. Links are preferably configured to illustrate various sets of applicable information, such as types of diseases, recommendations based on age, recommendations based on health condition, jurisdictional requirements, types of vaccines that prevent diseases, and vaccine safety.

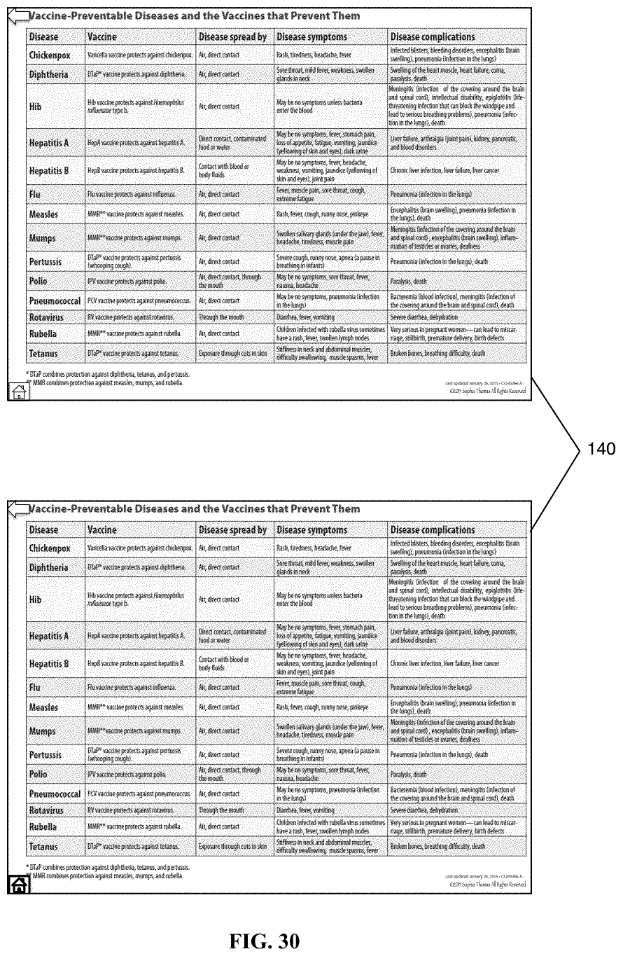

Referring to FIG. 30, snapshots of exemplary diagrams of windows 140 of an embodiments of an immunization tracker application of the system illustrate exemplary information related to one or more vaccine preventable diseases 152, in accordance with embodiments of the invention. As shown in FIG. 30, the processor 110 further provides to the client device 102 the information related to one or more diseases prevented by respective one or more vaccines, the information being received from the third party device 114. Further, in one embodiment, the information includes a disease name, a vaccine name, a disease symptom, a disease cause and a disease complication. In one embodiment, the disease name may be Diphtheria, Pertussis, Tetanus, Measles, Mumps, Rubella, Chickenpox, Haemophilus influenzae Type b (Hib), Hepatitis A, Hepatitis B, Flu, Polio, Pneumococcal, Rotavirus, or Tetanus.

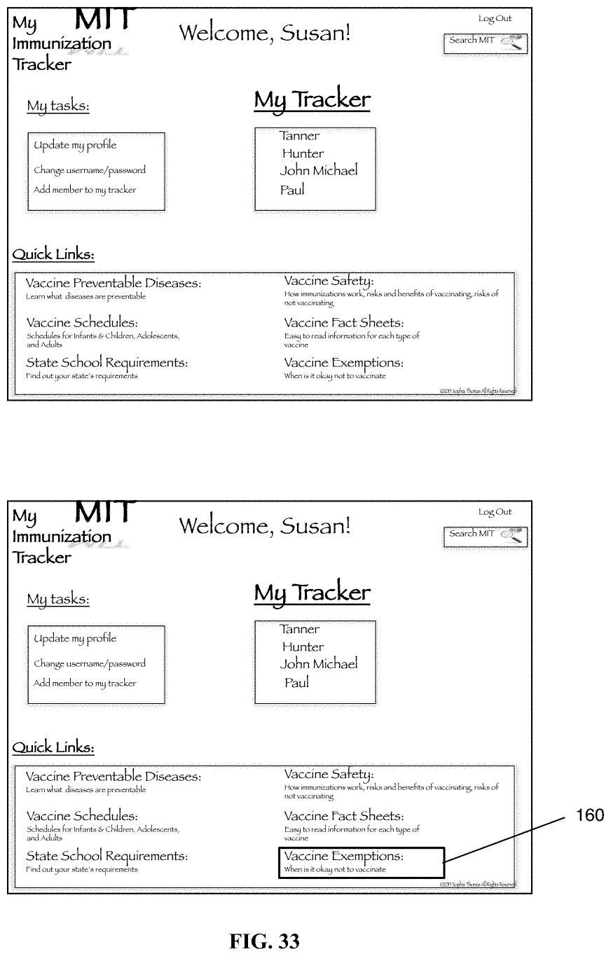

Referring to FIG. 33, embodiments of the invention provide information pertaining to vaccine exemptions 160, such as when it recommended or acceptable to refrain from receiving a vaccination.

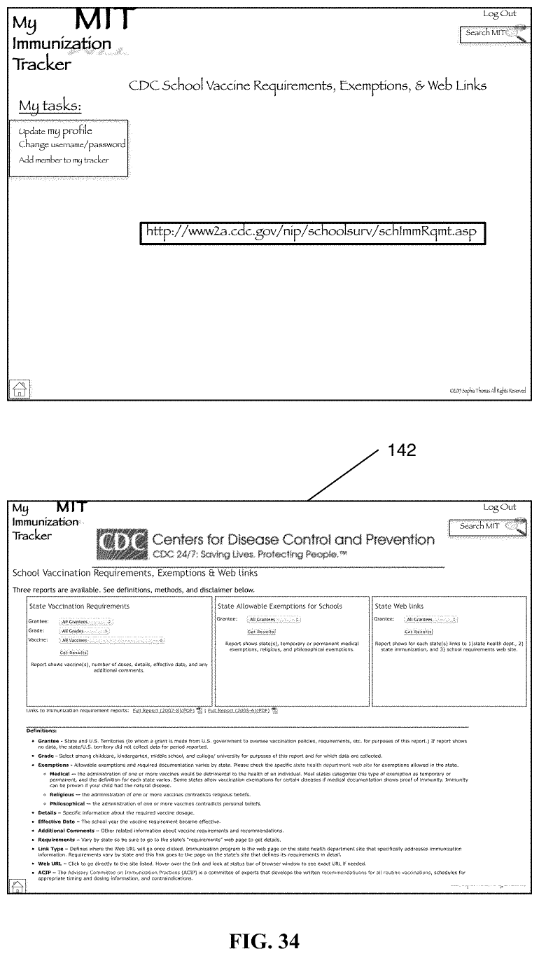

Referring to FIG. 34, that illustrates a snapshot of an exemplary window 142 of an immunization tracker application illustrating one or more vaccines exemptions 160, in accordance with embodiments of the invention. As shown in FIG. 34, the processor 110 further provides information related to one or more vaccines exemptions 160, received from the third party device 114, such as from CDC. In a preferred embodiment, the one or more vaccine exemptions 160 include temporary medical, permanent medical, religious and philosophical exemptions. Preferably, the one or more vaccine exemptions 160 are exemptions from State vaccination requirements 127. In yet another preferred embodiment, embodiments provide for State allowable exemptions for schools 162. In yet another preferred embodiment, embodiments generate and transit a report 164 to the client device 102 of a user 103 which shows each State(s) links to State Health Department, State immunization records, and school requirements.

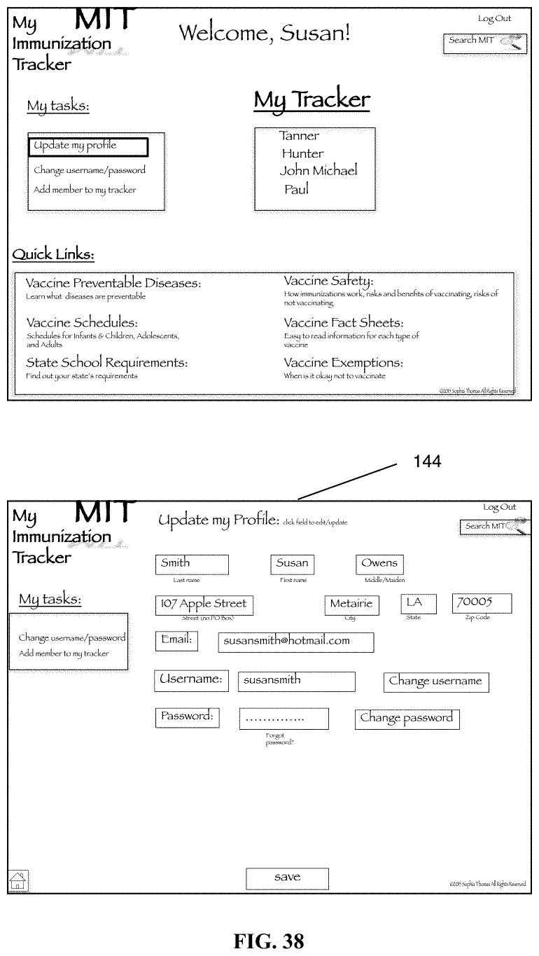

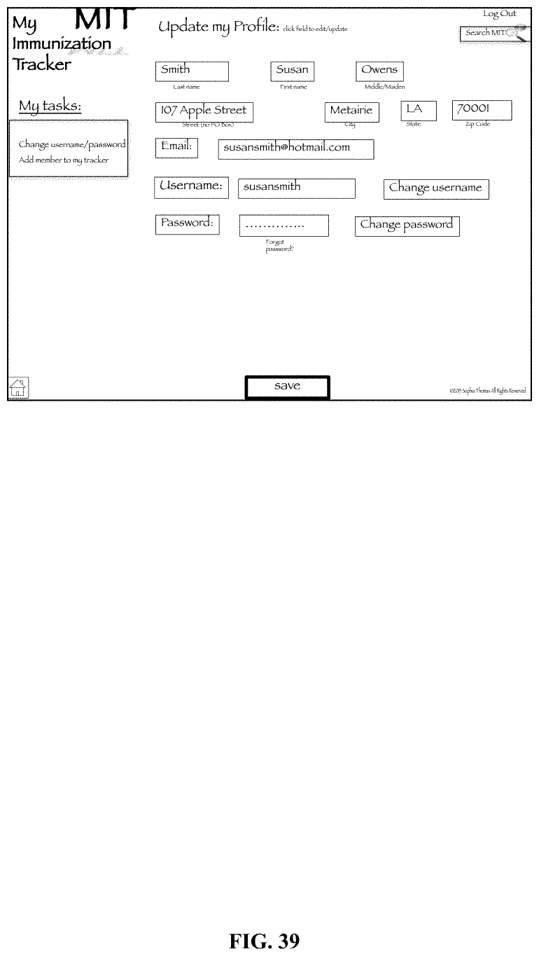

Referring to FIGS. 38-39, diagrams of exemplary windows of preferred embodiments of the immunization tracker application of the system disclosed herein show a prompt 144 to update and save profile information such as name, address, email address, username, and password.

In one embodiment, the requirement for vaccination is provided based on one or more states of health corresponding to the one or more respective family members 120.

In one embodiment, the processor 110 further updates profile information of the one or more family members 120 based upon recent updates. Preferably, the profile update comprises changes in first name, last name, middle name, street, city, state, zip code, email, user name and password.

The invention disclosed herein offers a number of advantages. The disclosed invention provides relevant information related to the one or more vaccines necessary for one's health management. Further, through embodiments disclosed herein, the present invention keeps track of regular vaccinations received by an individual, maintains the one or more records of the individual and provides alerts for vaccination to the individual on regular basis. Through preferred embodiments disclosed herein, the invention tracks a survival or disease status of a user 103 taking action toward achieving goals associated with improving health, based upon the at least one notification of vaccination recommendations 126.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data streams along a local network or a publicly accessible network such as the Internet.

It should also be understood that, unless specifically stated otherwise as apparent from the following discussion, it is appreciated that throughout the description, discussions utilizing terms such as “controlling” or “obtaining” or “computing” or “storing” or “receiving” or “determining” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The terms used herein are for the purpose of describing exemplary embodiments only and are not intended to be limiting. Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise. It will be further understood that the terms “comprises” or “comprising,” when used in this specification, do not preclude the presence or addition of one or more components, steps, operations, and/or elements other than a mentioned component, step, operation, and/or element.

This invention is susceptible to considerable variation within the spirit and scope of the appended claims.

The claimed invention is:

1. A computer implemented method for immunization tracking, comprising steps of:
receiving one or more immunization records from a state immunization registry, each immunization record corresponding to one or more respective persons;
receiving one or more schedules of vaccination requirements;
storing the one or more immunization records and schedules of vaccination requirements in a data repository;
receiving a request requesting notification of vaccination recommendations, from a client device;
analyzing, using a computer processor, the one or more immunization records by cross-referencing the one or more immunization records with the one or more schedules of vaccination requirements;
generating one or more notifications of vaccination recommendations in response to receiving the request; and
transmitting the one or more notifications of vaccination recommendations, to the client device.

2. The computer implemented method of claim 1, further comprising tracking a survival or disease status of a user taking action toward achieving goals associated with improving health based upon the at least one notification of vaccination recommendations.

3. The computer implemented method of claim 1, further comprising receiving one or more user vaccination records from the client device, wherein the step of analyzing the one or more immunization records includes cross-referencing the one or more user vaccination records with the one or more schedules of vaccination requirements, for generating the one or more notifications of vaccination recommendations in response to receiving the request.

4. The computer implemented method of claim 1, wherein the one or more schedules of vaccination requirements includes seasonal vaccination requirements, wherein the request requesting notification of vaccination recommendations is automatically triggered seasonally based upon the seasonal vaccination requirements.

5. The computer implemented method of claim 1, further comprising receiving one or more user date of birth records from the client device, wherein the step of analyzing the one or more immunization records includes cross-referencing the one or more user date of birth records with the one or more schedules of vaccination requirements, for generating the one or more notifications of vaccination recommendations in response to receiving the request.

6. The computer implemented method of claim 5, wherein the request requesting notification of vaccination recommendations is automatically triggered annually, based upon the date of birth.

7. The computer implemented method of claim 1, further comprising receiving one or more user life event records from the client device, wherein the step of analyzing the one or more immunization records includes cross-referencing the user life event records with the one or more schedules of vaccination requirements, for generating the one or more notifications of vaccination recommendations in response to receiving the request.

8. The computer implemented method of claim 7, wherein the request requesting notification of vaccination recommendations is automatically triggered based upon the life event.

9. The computer implemented method of claim 8, wherein the one or more schedules of vaccination requirements includes a Tdap vaccine requirement to protect newborn children from whooping cough, wherein the request requesting notification of vaccination recommendations is automatically triggered when the user life event records includes childbirth.

10. The computer implemented method of claim 1, further comprising receiving one or more geographical area outbreak records associated with vaccine preventable diseases from a third party device, wherein the step of analyzing the one or more immunization records includes cross-referencing the one or more geographical area outbreak records with the one or more schedules of vaccination requirements, for generating the one or more notifications of vaccination recommendations in response to receiving the request.

11. The computer implemented method of claim 10, further comprising receiving at least one user geographic location record as provided by a client device having an on-board GPS, wherein the request requesting notification of vaccination recommendations is automatically triggered when the one or more geographical area outbreak records includes the at least one user geographic location record.

12. The computer implemented method of claim 1, further comprising receiving one or more user allergy records from the client device, wherein the step of analyzing the one or more immunization records includes cross-referencing the one or more user allergy records with the one or more schedules of vaccination requirements, for generating the one or more notifications of vaccination recommendations in response to receiving the request.

13. The computer implemented method of claim 1, further comprising receiving one or more user health condition records from the client device, wherein the step of analyzing the one or more immunization records includes cross-referencing the one or more user health condition records with the one or more schedules of vaccination requirements, for generating the one or more notifications of vaccination recommendations in response to receiving the request.

14. The computer implemented method of claim 1, wherein the schedules of vaccination requirements include vaccination requirements for a specific country of destination of a user.

15. The computer implemented method of claim 1, wherein the schedules of vaccination requirements include vaccination requirements for a school, place of employment, State, or territory of a user.

16. The computer implemented method of claim 1, further comprising a step of transmitting information related to the one or more recommendations, from a third party device.

17. The computer implemented method of claim 16, wherein the information includes risks and benefits related to the one or more recommendations.

18. The computer implemented method of claim 1, further comprising a step of providing a schedule for vaccinations on the client device, the schedule for vaccinations being received from a third party device.

19. The computer implemented method of claim 18, wherein the schedule for vaccinations is designed to include schedules for one or more age groups.

20. The computer implemented method of claim 19, wherein the one or more age groups are selected from a group consisting of 0-6 years, 7-18 years, adults by age and adults by health condition, wherein the one or more recommendations includes contraindications.

21. The computer implemented method of claim 1, wherein the persons are one or more respective family members, further comprising a step of transmitting the one or more immunization records corresponding to the one or more respective family members, to the client device.

22. The computer implemented method of claim 1, wherein the one or more respective persons are family members, further comprising a step of receiving details of vaccinations already received by the one or more respective family members and adding the details to the one or more immunization records corresponding to the one or more respective family members.

23. The computer implemented method of claim 1, further comprising a step of updating the one or more immunization records of the one or more vaccines based on the one or more recommendations.

24. The computer implemented method of claim 1, further comprising a step of providing information related to one or more vaccines, from a third party device.

25. The computer implemented method of claim 1, further comprising a step of providing information related to one or more diseases prevented by respective one or more vaccines, from a third party device.

26. The computer implemented method of claim 25, wherein the information includes a disease name, a vaccine name, disease symptoms, disease causes and disease complications.

27. The computer implemented method of claim 1, further comprising a step of providing information related to one or more vaccine exemptions, from a third party device.

28. An electronic immunization tracking and notification system, comprising:
a memory unit configured to store machine readable instruct;
a processor operably connected with the memory unit, the processor obtaining the machine-readable instructions from the memory unit, and being configured by the machine-readable instructions to:
receive immunization records from a state immunization registry, each immunization record corresponding to one or more respective persons;
receive one or more schedules of vaccination requirements;
store the one or more immunization records and schedules of vaccination requirements in a data repository;
receive a request requesting notification of vaccination recommendations, from a client device;
analyze the one or more immunization records by cross-referencing the one or more immunization records with the one or more schedules of vaccination requirements;
generate one or more notifications of vaccination recommendations in response to receiving the request; and
transmit the one or more notifications of vaccination recommendations, to the client device.

29. The immunization tracking and notification system of claim 28, wherein each record of the one or more records, comprises one or more of, a name, a date of birth (DOB), a gender, a birth state, medical history and allergies.

30. The immunization tracking and notification system of claim 28, wherein the processor is further configured to provide at least one requirement for vaccination on the client device, the at least one requirement for vaccination being provided based on one or more states corresponding to the one or more respective family members.

31. The immunization tracking and notification system of claim 28, wherein the one or more recommendations is selected from a group consisting of medicine, therapy, counseling and vaccination.

32. The immunization tracking and notification system of claim 28, wherein the processor is further configured to notify the client device for vaccinations corresponding to the one or more respective family members.

33. The immunization tracking and notification system of claim 32, wherein the processor is further configured to notify the client device for upcoming vaccinations corresponding to the one or more respective family members.

34. The immunization tracking and notification system of claim 32, wherein the processor is further configured to notify the client device for due vaccinations corresponding to the one or more respective family members.

35. The immunization tracking and notification system of claim 32, wherein the processor is further configured to notify the client device for vaccinations based upon on one or more dates of birth (DOB), corresponding to the one or more respective family members.

36. The immunization tracking and notification system of claim 28, wherein the processor is further configured to transmit information related to the one or more recommendations, to the client device, received from a third party device.

37. The immunization tracking and notification system of claim 36, wherein the information comprises risks and benefits related to the one or more recommendations.

38. The immunization tracking and notification system of claim 28, wherein the processor is further configured to provide a schedule for vaccinations on the client device, the schedule for vaccinations being received from a third party device.

39. The immunization tracking and notification system of claim 38, wherein the schedule for vaccinations is designed to include schedules for one or more age groups.

40. The immunization tracking and notification system of claim 39, wherein the one or more age groups are selected from a group consisting of children aged 18 years or younger based on medical indications, adults 19-21 years, adults 22-26 years, adults 27-49 years, adults 50-64 years, adults 65+ years, and adults 18+ years by health condition.

41. The immunization tracking and notification system of claim 28, wherein the processor is further configured to transmit the one or more records corresponding to the one or more respective family members, to the client device.

42. The immunization tracking and notification system of claim 28, wherein the processor is further configured to provide information related to one or more diseases prevented by respective one or more vaccines from a third party device.

43. The immunization tracking and notification system of claim 42, wherein the information is selected from a group consisting of a disease name, a vaccine name, disease symptoms, disease causes and disease complications.

44. The immunization tracking and notification system of claim 28, wherein the processor is further configured to provide information related to the one or more vaccines exemptions, from a third party device.

45. The immunization tracking and notification system of claim 28, wherein the processor is further configured to receive details of vaccinations already received by the one or more family members and add the details to the one or more records corresponding to the one or more respective family members.

46. The immunization tracking and notification system of claim 28, wherein the processor is further configured to update the one or more records of the one or more vaccines based on the one or more recommendations.

47. The immunization tracking and notification system of claim 28, wherein the processor is further configured to track a survival or disease status of a user taking action toward achieving goals associated with improving health based upon the at least one notification of vaccination recommendations.
